(19) United States
(12) Patent Application Publication
(10) Pub. No.: US 2026/0034052 A1
(43) Pub. Date: Feb. 5, 2026

(54) SYSTEMS, COMPOSITIONS, AND METHODS RELATED TO INJECTABLE HYDROGELS

(71) Applicant: Massachusetts Institute of Technology, Cambridge, MA (US)

(72) Inventors: Patrick S. Doyle, Sudbury, MA (US); Talia Zheng, Cambridge, MA (US)

(73) Assignee: Massachusetts Institute of Technology, Cambridge, MA (US)

(21) Appl. No.: 19/246,147

(22) Filed: Jun. 23, 2025

Related U.S. Application Data
(60) Provisional application No. 63/663,260, filed on Jun. 24, 2024.

(51) Int. Cl. A61K 9/00; A61K 9/06; A61K 9/14; A61K 45/06

(52) U.S. Cl. CPC A61K 9/0019; A61K 9/06; A61K 9/146; A61K 45/06

(57) ABSTRACT

Systems, compositions, and methods related to injectable hydrogels are generally described. In some embodiments, a composition comprises a first polymer and a plurality of particles comprising a second polymer capable of forming a hydrogel at elevated temperatures, such as body temperature. The plurality of particles (e.g., hydrogel particles) may comprise an active substance, such as a biological material and/or a therapeutic agent. Together, the first polymer and the second polymer may interact with each other (e.g., the first polymer may interpenetrate the second polymer, and/or the first polymer may cross-link with the second polymer) at elevated temperatures to form a hydrogel. Interactions between the first and second polymer, and/or between hydrophobic domains thereof, may facilitate the relatively slow and/or controlled release of the active substance. In some embodiments, the plurality of particles may have advantageously high loadings of biological materials (e.g., antibodies, proteins, peptides).

### RELATED APPLICATIONS

This application claims priority under 35 U.S.C. § 119(e) to U.S. Provisional Patent Application No. 63/663,260, filed Jun. 24, 2024, and entitled “Thermally-Gelling Injectable Hydrogel Composites for Therapeutic Delivery,” which is incorporated herein by reference in its entirety for all purposes.

### TECHNICAL FIELD

Systems, compositions, and methods related to hydrogels are generally described.

### BACKGROUND

Hydrogels are water-permeable networks of crosslinked polymers frequently employed in injectable therapeutics due to their softness and biocompatibility. Release of drug molecules from hydrogel-based drug delivery systems often occur in a rapid, uncontrolled manner due to the high permeability of the network, resulting in what is known as the ‘burst release’ effect. Sustained, controlled release of therapeutics is desired for many reasons, such as improving the efficacy of the therapy, preventing dose-related toxicities, and reducing the frequency of dosages.

### SUMMARY

Systems, compositions, and methods related to hydrogels are generally described. The subject matter of the present disclosure involves, in some cases, interrelated products, alternative solutions to a particular problem, and/or a plurality of different uses of one or more systems and/or articles.

In one aspect of the present disclosure, compositions are provided.

In some embodiments, the composition comprises: a first polymer capable of gelling when exposed to a temperature greater than or equal to 20 degrees Celsius; and a plurality of particles comprising a second polymer and a biological material, wherein: the first polymer is capable of interpenetrating with the second polymer when the composition is exposed to a temperature of greater than or equal to 20 degrees, and the plurality of particles comprises the biological material in an amount greater than or equal to 20 wt %.

In some embodiments, the composition comprises: a first polymer capable of gelling when exposed to a temperature greater than or equal to 20 degrees Celsius; and a plurality of particles comprising a second polymer and an active substance, wherein: the first polymer is capable of interpenetrating with the second polymer when the composition is exposed to a temperature of greater than or equal to 20 degrees, the plurality of particles comprises the active substance in an amount greater than or equal to 20 wt %, the composition is configured such that the active substance is released from the plurality of particles at a particular initial average rate as determined by the first 24 hours of release, and the composition the active substance is released at an average rate of at least 20% over a 24 hour period after the first 24 hours of release.

In some embodiments, the composition comprises a first polymer comprising a first hydrophobic domain, the first polymer capable of gelling when exposed to a temperature greater than or equal to 20 degrees Celsius; and a plurality of particles comprising a second polymer and an active substance, wherein: the second polymer comprises a second hydrophobic domain, the first hydrophobic domain is capable of interacting with the second hydrophobic domain when the composition is exposed to a temperature of greater than or equal to 20 degrees, the first polymer is capable of coupling with the second polymer, the composition is configured such that the active substance is released from the plurality of particles at a particular initial average rate as determined by the first 24 hours of release, and the composition the active substance is released at an average rate of at least 20% over a 24 hour period after the first 24 hours of release.

Other advantages and novel features of the present disclosure will become apparent from the following detailed description of various non-limiting embodiments of the disclosure when considered in conjunction with the accompanying figures. In cases where the present specification and a document incorporated by reference include conflicting and/or inconsistent disclosure, the present specification shall control.

### BRIEF DESCRIPTION OF THE DRAWINGS

Non-limiting embodiments of the present disclosure will be described by way of example with reference to the accompanying figures, which are schematic and are not intended to be drawn to scale unless otherwise indicated. In the figures, each identical or nearly identical component illustrated is typically represented by a single numeral. For purposes of clarity, not every component is labeled in every figure, nor is every component of each embodiment of the disclosure shown where illustration is not necessary to allow those of ordinary skill in the art to understand the disclosure. In the figures:

FIG. 1 is a schematic depicting a dual-network hydrogel design, compositing high-concentration antibody-loaded alginate microparticles with thermogelling methylcellulose polymer to result in a thermo-gelling composite hydrogel with complex inter-network interactions, according to some embodiments.

FIG. 2A shows brightfield microscopy images, according to some embodiments.

FIG. 2B shows particle size distributions of synthesized antibody-loaded alginate microparticles with (left to right) 0%, 3%, and 6% degree of substitution, according to some embodiments.

FIGS. 2C-2D show small amplitude oscillatory shear temperature sweep data for (FIG. 2C) 2% w/v alginate solutions with varying degree of substitution and (FIG. 2D) 4% w/v methylcellulose and 1% w/v alginate solutions with varying degree of substitution. (⋅) denotes storage modulus (G′) and (°) denotes loss modulus (G″), according to some embodiments.

FIG. 2E shows the swelling ratio (Qs) for blank (no ASD) alginate microparticles synthesized using 2% w/v alginate with varying degree of substitution, according to some embodiments.

FIGS. 3A-3D show profiles of in vitro release tests performed in simulated bodily fluid, with dashed lines fitted to the Weibull model, for (FIGS. 3A-3B) ASD (amorphous solid antibody without alginate) and ASD pre-gel (solid antibody with 2% w/v uncross-linked alginate) formulations (FIG. 3A) without methylcellulose (R2=0.99-0.999) and (FIG. 3B) with the addition of 4% w/v methylcellulose (R2=0.98-0.99), and (FIGS. 3C-3D) ASD-laden alginate (2% w/v) microparticles (FIG. 3C) suspended in buffer (R2=0.99-0.991) and (FIG. 3D) suspended in buffer with 4% w/v methylcellulose (R2=0.98-0.995), according to some embodiments.

FIG. 4A shows a schematic of injection force testing set-up. Injection force (N) versus distance traveled by syringe plunger (mm) for formulations tested at (FIG. 4B) 25 μL/s (˜1.3 mm/s) and (FIG. 4C) 150 μL/s (˜8.3 mm/s), according to some embodiments.

FIG. 5 shows an oxidation-reductive amination reaction scheme for hydrophobic modification of alginate, according to some embodiments.

FIG. 6 shows the steps of the formulation process for the alginate-methylcellulose composite hydrogel, according to some embodiments.

FIG. 7 shows the solubility of IgG (in mg/mL) from alginate (0%, 3%, or 6% degree of substitution) particles suspended in methylcellulose solution, according to some embodiments.

FIGS. 8A-8E show UV traces from size exclusion chromatography of released IgG from (FIG. 8A) control sample, (FIG. 8B) alginate particles, 0% d.s., (FIG. 8C) alginate particles, 6% d.s., (FIG. 8D) alginate particles in methylcellulose, 0% d.s., and (FIG. 8E) alginate particles in methylcellulose, 6% d.s., according to some embodiments.

FIGS. 9A-9B show the small amplitude oscillatory shear (FIG. 9A) temperature sweep data for 4% w/v methylcellulose and 1% w/v alginate (0% d.s.) solution with 2 cycles of heating, and (FIG. 9B) time sweep data for 4% w/v methylcellulose and 1% w/v alginate (0% d.s.) solution with 2 cycles of heating and cooling. (⋅) denotes storage modulus (G′), (°) denotes loss modulus (G″), and - - - denotes temperature, according to some embodiments.

FIGS. 10A-10B show the profiles of in vitro release tests performed in simulated bodily fluid, with dashed lines fitted to the Weibull model for the averaged data, for ASD-laden alginate (2% w/v) microparticles (FIG. 10A) suspended in buffer with 0% (R2-0.965) and 3% (R2=0.991) degree of alginate substitution, and (FIG. 10B) suspended in buffer with 4% w/v methylcellulose with 0% (R2=0.986) and 3% (R2=0.991) degree of alginate substitution, according to some embodiments.

FIG. 11 shows an image of the set-up for the injectability tests, displayed at the beginning of a test, according to some embodiments.

FIG. 12 shows the hydrogel composite consists of a thermally gelling polymer 1 and crosslinked hydrogel particles from polymer 2 which encapsulates the drug cargo, according to some embodiments.

FIG. 13A shows the chemical structure of methylcellulose (polymer 1), according to some embodiments.

FIG. 13B shows the chemical structure of alkylated alginate (polymer 2), with x unmodified repeated units and y modified repeated units, according to some embodiments.

FIGS. 14A-14B show in vitro release data in simulated bodily fluid at 37° C. for solid IgG in FIG. 14A) alginate particles and pre-gel (un-crosslinked alginate) and FIG. 14B) alginate particles and pre-gel composited with methylcellulose, according to some embodiments.

FIGS. 15A-15B show G′ (closed circles) and G″ (open circles) under small amplitude oscillatory shear (0.1% strain, 10 rad/s frequency) from 20° C. to 70° C., for FIG. 15A) unmodified (Alg) and modified (AAlg) solutions at 2% w/v polymer concentration and FIG. 15B) methylcellulose (MC) solutions, at 4% w/v, without or with either unmodified alginate or modified alginate at 1% w/v, according to some embodiments.

### DETAILED DESCRIPTION

In the last decade, there has been progress in the treatment of cancer and auto-immune diseases through the administration of biologics, specifically antibody drugs. These antibodies are often formulated as liquids at low concentrations and injected intravenously; however, IV infusions typically require hospital/clinic care and are burdensome for both patients and providers. Subcutaneous (SC) injection is a more preferred delivery format and can also allow for self-administration and home-based care. In SC delivery, the total injection volume is limited (typically 2 mL or less), making high-concentration antibody solutions (>100 mg/mL) desirable. However, such solutions are viscous due to self-association among the antibodies, and therefore challenging to process and deliver. SC delivery as an alternative to IV has been emerging as part of the paradigm shift towards patient-centric clinical practice and out-of-clinic care, making high-concentration antibody formulations desirable for current and future developments in the therapeutic landscape. The high viscosity and instability can be addressed by formulating antibodies as amorphous solid dispersions (ASDs), which can be packed to high concentrations. Further, the antibody ASDs are encapsulated in alginate microparticles, which are biocompatible, shear-thinning materials that allow the solid antibodies to be easily injected. With this approach, stable, high-concentration protein suspensions can be formulated. However, while this approach is promising, the permeability and fast swelling of the alginate hydrogel mesh leads to burst release of the antibody, which can reduce the dosage efficacy as well as potentially lead to systemic side effects. These effects are especially pronounced at high concentrations, as hydrogels typically lead to a larger burst release in the case of high drug loadings. Therefore, there is a need to develop high-concentration antibody formulations that allow for relatively consistent and sustained delivery, which are preferred for long-term efficacy and case of use.

Thermoresponsive polymers can be used to achieved sustained release in hydrogel drug delivery systems and have been previously investigated for SC-injectable biologics. These polymers are liquid in solution at room temperature and gel at body temperature, thus slowing diffusion from the hydrogel and erosion thereof. Composite hydrogels are also possible. For example, polymer micro-or nanoparticles may be embedded within a thermo-gelling matrix that eliminates the burst release from the particles alone. Notably, typical injectable formulations in these systems have been limited to low drug concentrations (<1-50 mg/mL). Methylcellulose (MC) is a thermoresponsive polysaccharide which can suppress burst release due to its ability to form a depot at body temperature and has been shown to be biocompatible and non-toxic in the SC environment. An increase in temperature may cause fibril formation as well as the association of hydrophobic domains leading rise to the gel network structure. Methylcellulose is also may form semi-interpenetrating networks with alginate, due to MC's ability to thermally gel through hydrophobic associations and alginate's native ionic cross-linking as well as hydrogen bonding between the two networks. As described herein, antibody-laden alginate microparticles were combined with a methylcellulose thermogel to suppress burst release and instead allow for sustained release from the particles while maintaining the advantages of hydrogel encapsulation of the highly concentrated antibodies, such as injectability. The inter-network polymer interactions may be tuned through chemical modification of alginate to tune the release behavior of the composite hydrogel.

Systems, compositions, and methods related to injectable hydrogels are generally described. In some embodiments, a composition comprises a first polymer and a second polymer capable of forming a hydrogel at elevated temperatures, such as body temperature. The second polymer may be present in a plurality of particles (e.g., hydrogel particles). For example, the second polymer may comprise the second polymer and an active substance, such as a biological material and/or a therapeutic agent. Together, the first polymer and the second polymer may interact with each other (e.g., the first polymer may interpenetrate the second polymer, and/or the first polymer may cross-link with the second polymer) at elevated temperatures to form a hydrogel. The formation of the gel may occur in situ once injected into a subject. For example, the composition may be loaded into a fluidic delivery device (e.g., a syringe) and administered to subject. After administration, the composition may be exposed to an environment having elevated temperatures (e.g., approximately 37 degrees Celsius) thereby gelling the composition. The active substance, in a controlled and sustained manner, may be released in the subject over a period of hours, days, weeks, and/or months. Interactions between the first and second polymer, and/or between hydrophobic domains thereof, may facilitate the relatively slow and/or controlled release of the active substance. In some embodiments, the plurality of particles may have advantageously high loadings of biological materials (e.g., antibodies, proteins, peptides).

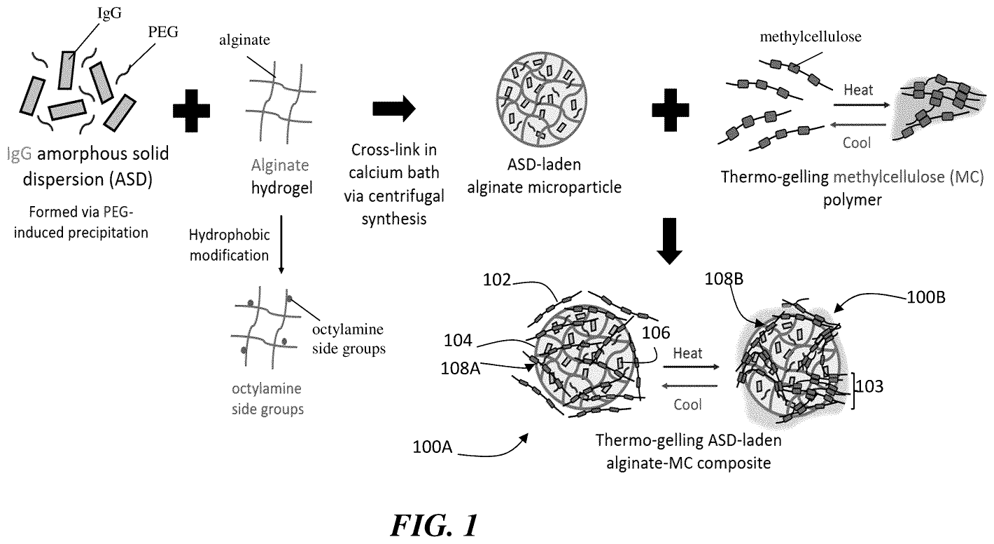

In some embodiments, the composition comprises a first polymer. In some embodiments, the first polymer may be a thermoresponsive polymer (e.g., a thermogelling polymer). Thermoresponsive polymers may undergo a phase transition in response to a temperature change. In some cases, the phase of the polymer may transition from a liquid at a first temperature to a gel at a second temperature that is greater than the first temperature. For instance, some thermoresponsive polymers may be a liquid at room temperature, but when exposed to an elevated temperature, such as body temperature (e.g., 37 degrees Celsius), the polymer may form a gel. For example, as shown in FIG. 1, composition 100A comprises first polymer 102 which is thermoresponsive. When composition 100A is heated to above the lower critical solution temperature of first polymer 102, first polymer 102 may form gel 103 in composition 100B. Thermoresponsive polymers are especially desirable in drug delivery applications, because such polymers may have a relatively low viscosity at room temperature which facilitates its administration (e.g., injected) at room temperature and a relatively high viscosity when in the subject. Moreover, after administration of the polymer, migration of polymer within the subject may then be limited due to the relatively high viscosity of the polymer in the gel phase. To determine whether a polymer is a suitable thermoresponsive polymer for compositions described herein, one may heat the polymer, or a mixture comprising the polymer, to a temperature (e.g., 37 degrees Celsius) and observe whether gelation occurs. Gelation may be indicated by a change in rheology of the polymer, such an increase in the viscosity, shear modulus, and/or loss modulus of the polymer. Gelation may therefore be monitored using a rheometer or the like.

In some embodiments, the first polymer may be any of a variety of thermoresponsive polymers. In some embodiments, the first polymer comprises hydroxypropylmethylcellulose, carboxymethylcellulose, chitosan, poly(N-isopropylacrylamide), a polycaprolactone copolymer, a polycarbonate, a poloxamer, and/or a PEG-PLGA copolymer. Other thermoresponsive polymer may also be included. In some embodiments, the first polymer is capable of forming a hydrogel by itself or in conjunction with another polymer (e.g., the second polymer).

In some embodiments, the first polymer comprises one or more hydrophobic domains. For example, the first polymer may comprise a first hydrophobic domain capable of interacting with one or more hydrophobic domains of the second polymer. For instance, the first polymer may be alkylated such that hydrophobic alkyl groups, forming the first hydrophobic domain, may interact with hydrophobic groups of the second polymer. Such interaction may increase the viscosity of the composition at elevated temperatures thereby reducing the rate at which the active substance can migrate out of the composition. In some embodiments, the first polymer may be modified with one or more hydrophobic domains. In some embodiments, the first polymer can be alkylated such that the first polymer comprises an alkyl group comprising at least 1 carbon atom, at least 2 carbon atoms, at least 3 carbon atoms, at least 4 carbon atoms, at least 5 carbon atoms, at least 6 carbon atoms, at least 7 carbon atoms, at least 8 carbon atoms, at least 9 carbon atoms, at least 10 carbon atoms, or more.

In some embodiments, the first polymer is capable of undergoing gelation at elevated temperatures. For example, polymer chains of the first polymer may at least partially entangle with each other at elevated temperature which may increase the viscosity of the first polymer and form a gel (e.g., a hydrogel). In some embodiments, the first polymer is capable of gelling when exposed to a temperature greater than or equal to 20 degrees Celsius, greater than or equal to 22.5 degrees Celsius, greater than or equal to 25 degrees Celsius, greater than or equal to 27.5 degrees Celsius, greater than or equal to 30 degrees Celsius, greater than or equal to 32.5 degrees Celsius, greater than or equal to 35 degrees Celsius, greater than or equal to 37 degrees Celsius, greater than or equal to 37.5 degrees Celsius, or greater than or equal to 40 degrees Celsius. In some embodiments, the first polymer is capable of gelling when exposed to a temperature less than or equal to 40 degrees Celsius, less than or equal to 37.5 degrees Celsius, less than or equal to 37 degrees Celsius, less than or equal to 35 degrees Celsius, less than or equal to 32.5 degrees Celsius, less than or equal to 30 degrees Celsius, less than or equal to 27.5 degrees Celsius, less than or equal to 25 degrees Celsius, less than or equal to 22.5 degrees Celsius, or less than or equal to 20 degrees Celsius. Combinations of these ranges are possible (e.g., greater than or equal to 20 degrees Celsius and less than or equal to 40 degrees Celsius). Other ranges are possible.

In some embodiments, the composition comprises a second polymer. For example, as shown in FIG. 1, composition 100A comprises second polymer 104 in the form of a particle. In some embodiments, the first polymer may be coupled with the second polymer by chemical interactions and/or by interpenetrating (e.g., entangled) polymer chains. For example, in some embodiments, a polymer backbone of the first polymer and a polymer backbone the second polymer are coupled via a bond such as an ionic bond, a covalent bond, a hydrogen bond, Van der Waals interactions, and/or the like. The covalent bond may be, for example, carbon-carbon, carbon-oxygen, oxygen-silicon, sulfur-sulfur, phosphorus-nitrogen, carbon-nitrogen, metal-oxygen, or other covalent bonds. The hydrogen bond may be, for example, between hydroxyl, amine, carboxyl, thiol, and/or similar functional groups. An example of this is shown in FIG. 1. Composition 100B, which is present at an elevated temperature above the lower critical solution temperature of composition 100B, comprises first polymer 102 that is interpenetrated with second polymer 104. Region 108B indicates a point of entanglement between first polymer 102 and second polymer 104. Region 108B is associated a point of interpenetration between first polymer 102 and second polymer 104. In some embodiments, the first polymer is capable of coupling the second polymer. For instance, the first and second polymer may have moieties that may interact with each (e.g., covalently and/or noncovalently) as described above. In some cases, it may be possible to determine whether a first polymer has interpenetrated a second polymer based on the rheology of the resulting polymeric network (e.g., the composition). For example, if, at a particular temperature (e.g., 37 degrees Celsius), the viscosity of the polymeric network is greater than the viscosity of the first polymer and the viscosity of the second polymer, then the first polymer and second polymer may be interpenetrated. Changes in the storage modulus and/or the loss modulus may also serve as an indicator that interpenetration of polymers may be present. For example, if, at a particular temperature (e.g., 37 degrees Celsius), the storage modulus of the polymeric network is greater than the storage modulus of the first polymer and the storage modulus of the second polymer, then the first polymer and second polymer may be interpenetrated. It should be appreciated that changes in viscosity, storage modulus, and/or loss modulus may also be due, at least in part, to a myriad of other factors, but one or more of such changes may nevertheless serve as an indicator that interpenetration of polymers may be present.

In some embodiments, the first polymer is capable of interpenetrating with the second polymer. For instance, the first polymer may be physically entangled with the second polymer. When the first polymer is entangled with the second polymer, a polymeric matrix (e.g., an interpenetrated polymer matrix) comprising the first and second polymer may be formed. Physical entanglements may involve interactions between the first and second polymer, including but not limited to secondary bonds (e.g., van der waals interactions, hydrogen bonding, and/or London dispersion forces). For example, as shown in FIG. 1, first polymer 102 is physically entangled with second polymer 104 at region 108A. As composition 100A is at a temperature lower than the lower critical solution temperature, such entanglements may form via diffusion and/or movement of first polymer 102 into second polymer 104. Active substance in the plurality of particle may then migrate and/or be released from the composition at a relatively slow rate due, at least in part, to such physical entanglements.

In some embodiments, the composition (e.g., a hydrogel) comprises an interpenetrating polymer network comprising at least a first polymer and a second polymer interpenetrating each other. In certain embodiments, the first polymer comprises at least a first cross-link moiety. For example, the interpenetrating polymer network may be formed by mixing two or more monomers (or oligomers, or polymers, or prepolymers) and one or more crosslinking reagents (e.g., a bifunctional monomer, a polyfunctional monomer) such that a first monomer reacts forming a first polymer comprising a first crosslink moiety (e.g., comprising at least a portion of a first crosslinking reagent) and/or a second monomer reacts forming a second polymer comprising a second crosslink moiety (e.g., comprising at least a portion of a second crosslinking reagent). In some embodiments, additional crosslinking may occur between the first and second polymers such that an interpenetrating network is formed between the first and second polymers. For example, the first polymer may be modified to the second polymer and/or the second polymer may be modified to crosslink with the first polymer. The polymer network resulting from the crosslinking of the first polymer and the second polymer may comprise the crosslink moiety.

In some embodiments, the first polymer is capable of interpenetrating with the second polymer when the composition is exposed to an elevated temperature. In some embodiments, the first polymer is capable of interpenetrating with the second polymer when the composition is exposed to a temperature greater than or equal to 20 degrees Celsius, greater than or equal to 22.5 degrees Celsius, greater than or equal to 25 degrees Celsius, greater than or equal to 27.5 degrees Celsius, greater than or equal to 30 degrees Celsius, greater than or equal to 32.5 degrees Celsius, greater than or equal to 35 degrees Celsius, greater than or equal to 37 degrees Celsius, greater than or equal to 37.5 degrees Celsius, or greater than or equal to 40 degrees Celsius. In some embodiments, the first polymer is capable of interpenetrating with the second polymer when the composition is exposed to a temperature less than or equal to 40 degrees Celsius, less than or equal to 37.5 degrees Celsius, less than or equal to 37 degrees Celsius, less than or equal to 35 degrees Celsius, less than or equal to 32.5 degrees Celsius, less than or equal to 30 degrees Celsius, less than or equal to 27.5 degrees Celsius, less than or equal to 25 degrees Celsius, less than or equal to 22.5 degrees Celsius, or less than or equal to 20 degrees Celsius. Combinations of these ranges are possible (e.g., greater than or equal to 20 degrees Celsius and less than or equal to 40 degrees Celsius). Other ranges are possible.

In some embodiments, the first polymer is capable of crosslinking with the second polymer. For example, the first polymer may covalently and/or non-covalently interact with the second polymer such that a polymeric network (e.g., a hydrogel) is formed comprising the first and second polymers. In some cases, such crosslinking may occur at elevated temperatures (e.g., body temperature). For instance, when the composition is injected into a subject, the composition will be exposed to environment having an elevated temperature. Such exposure may facilitate crosslinking between the first and second polymers. The elevated temperature, as described elsewhere in the disclosure, may also facilitate gelation of the composition. In some embodiments, the first polymer is capable of cross-linking with the second polymer such that, when exposed to a temperature greater than or equal to 20 degrees Celsius, greater than or equal to 25 degrees Celsius, greater than or equal to 30 degrees Celsius, greater than or equal to 35 degrees Celsius, or greater than or equal to 40 degrees Celsius. In some embodiments, the first polymer is capable of cross-linking with the second polymer such that, when exposed to a temperature less than or equal to 40 degrees Celsius, less than or equal to 35 degrees Celsius, less than or equal to 30degrees Celsius, less than or equal to 25 degrees Celsius, or less than or equal to 20 degrees Celsius. Combinations of these ranges are possible (e.g., greater than or equal to 20 degrees Celsius and less than or equal to 40 degrees Celsius). Other ranges are possible.

In some cases, the second polymer comprises one or more hydrophobic domains. For instance, the polymer backbone of the second polymer may be modified to have a second hydrophobic domain, such as an alkyl group. When combined with the first polymer in a mixture, the hydrophobic domains of the first polymer may interact with the hydrophobic domains of the second polymer. This interaction may allow active substances (e.g., biologics and/or pharmaceuticals) within the plurality of particles to be released in a sustained and/or controlled manner. Moreover, such interaction may increase the viscosity of the composition at elevated temperatures (e.g., after gelation) thereby reducing the rate at which the active substance can migrate out of the composition. In some embodiments, the second polymer may be modified with one or more hydrophobic domains. In some embodiments, the second polymer can be alkylated such that the second polymer comprises an alkyl group comprising at least 1 carbon atom, at least 2 carbon atoms, at least 3 carbon atoms, at least 4 carbon atoms, at least 5 carbon atoms, at least 6 carbon atoms, at least 7 carbon atoms, at least 8 carbon atoms, at least 9 carbon atoms, at least 10 carbon atoms, or more.

In some embodiments, the first hydrophobic domain may be capable of interacting the second hydrophobic domain at elevated temperatures. In some embodiments, the first hydrophobic domain is capable of interacting with the second hydrophobic domain when the composition is exposed to a temperature of greater than or equal to 20 degrees Celsius, greater than or equal to 22.5 degrees Celsius, greater than or equal to 25 degrees Celsius, greater than or equal to 27.5 degrees Celsius, greater than or equal to 30 degrees Celsius, greater than or equal to 32.5 degrees Celsius, greater than or equal to 35 degrees Celsius, greater than or equal to 37 degrees Celsius, greater than or equal to 37.5 degrees Celsius, or greater than or equal to 40 degrees Celsius. In some embodiments, the first hydrophobic domain is capable of interacting with the second hydrophobic domain when the composition is exposed to a temperature of less than or equal to 40 degrees Celsius, less than or equal to 37.5 degrees Celsius, less than or equal to 37 degrees Celsius, less than or equal to 35 degrees Celsius, less than or equal to 32.5 degrees Celsius, less than or equal to 30 degrees Celsius, less than or equal to 27.5 degrees Celsius, less than or equal to 25 degrees Celsius, less than or equal to 22.5 degrees Celsius, or less than or equal to 20 degrees Celsius. Combinations of these ranges are also possible (e.g., greater than or equal to 20 degrees Celsius and less than or equal to 40 degrees Celsius). Other ranges are possible.

In some embodiments, the second polymer may comprise any of a variety of suitable polymers. In some embodiments, the second polymer is capable of forming a hydrogel by itself or in conjunction with another polymer (e.g., the first polymer). In some embodiments, the second polymer comprises the second polymer comprises alginate, polyethylene glycol, gelatin and/or agarose. Other polymers may be used, including those capable of forming a hydrogel.

In some embodiments, the composition comprises a plurality of polymers comprising the second polymer. The plurality of particles may be dispersed in the first polymer and/or a network thereof. For example, in some cases, the plurality of particles may be dispersed in a mixture comprising the first polymer. After the mixture is exposed to a temperature greater than or equal to the critical solution temperature (CST), the mixture may form a gel at which point the particles may be interpenetrated with the first polymer. After the mixture reaches and/or exceeds the CST, polymer chains of the first polymer may couple and/or otherwise bond to the polymer chains of the second polymer such that the second polymer of the plurality of particles is interpenetrated with the first polymer. In some cases, the first polymer may diffuse into the plurality of particles and entangle themselves in the first polymer. For instance, the polymer chains of the first polymer may at least partially penetrate at least some of the plurality of particles.

The plurality of particles may have any of a variety of suitable sizes. In some embodiments, the plurality of particles have an average maximum dimension of greater than or equal to 5 micrometers, greater than or equal to 10 micrometers, greater than or equal to 50 micrometers, greater than or equal to 100 micrometers, greater than or equal to 150 micrometers, greater than or equal to 200 micrometers, greater than or equal to 250 micrometers, greater than or equal to 300 micrometers, greater than or equal to 350 micrometers, greater than or equal to 400 micrometers, greater than or equal to 450 micrometers, or greater than or equal to 500 micrometers. In some embodiments, the plurality of particles have an average maximum dimension of less than or equal to 500 micrometers, less than or equal to 450 micrometers, less than or equal to 400 micrometers, less than or equal to 350 micrometers, less than or equal to 300 micrometers, less than or equal to 250 micrometers, less than or equal to 200 micrometers, less than or equal to 150 micrometers, or less than or equal to 100 micrometers. Combinations of these ranges are possible (e.g., greater than or equal to 100 micrometers and less than or equal to 500 micrometers). Other ranges are possible.

According to some embodiments, the systems, articles, and methods described herein are compatible with one or more therapeutic, diagnostic, and/or enhancement agents, such as drugs, nutrients, microorganisms, in vivo sensors, and tracers. In some embodiments, the active substance, is a therapeutic, nutraceutical, prophylactic or diagnostic agent. The active substance may be entrapped within the polymeric matrix (e.g., the second polymer) of the plurality of particles or may be directly attached to one or more atoms in the polymeric matrix (e.g., the second polymer) through a chemical bond. In certain embodiments, the active substance is covalently bonded to the polymeric matrix of the second polymer. For example, as shown in FIG. 1, active substance 106 is entrapped in the particle comprising second polymer 104. Active substance 106 may navigate around regions 108A and 108B to exit the particle and be transported throughout the subject. In some embodiments, the active substance may be positioned in the plurality of particles during the fabrication of the plurality of the particles. For example, when forming the plurality of particles from the second polymer (e.g., alginate as described in the Examples), the active substance may be introduced into the particles during such formation.

Active substances can include, but are not limited to, any synthetic or naturally-occurring biologically active compound or composition of matter (e.g., a biological material) which, when administered to a subject (e.g., a human or nonhuman animal), induces a desired pharmacologic, immunogenic, and/or physiologic effect by local and/or systemic action. For example, useful or potentially useful within the context of certain embodiments are compounds or chemicals traditionally regarded as drugs, vaccines, and biopharmaceuticals. Certain such agents may include molecules such as proteins, peptides, hormones, nucleic acids, gene constructs, and/or other biologics for use in therapeutic, diagnostic, and/or enhancement areas, including, but not limited to medical or veterinary treatment, prevention, diagnosis, and/or mitigation of disease or illness (e.g., HMG co-A reductase inhibitors (statins) like rosuvastatin, nonsteroidal anti-inflammatory drugs like meloxicam, selective serotonin reuptake inhibitors like escitalopram, blood thinning agents like clopidogrel, steroids like prednisone, antipsychotics like aripiprazole and risperidone, analgesics like buprenorphine, antagonists like naloxone, montelukast, and memantine, cardiac glycosides like digoxin, alpha blockers like tamsulosin, cholesterol absorption inhibitors like ezetimibe, metabolites like colchicine, antihistamines like loratadine and cetirizine, opioids like loperamide, proton-pump inhibitors like omeprazole, antiviral agents like entecavir, antibiotics like doxycycline, ciprofloxacin, and azithromycin, anti-malarial agents, and synthroid/levothyroxine); substance abuse treatment (e.g., methadone and varenicline); family planning (e.g., hormonal contraception); performance enhancement (e.g., stimulants like caffeine); and nutrition and supplements (e.g., protein, folic acid, calcium, iodine, iron, zinc, thiamine, niacin, vitamin C, vitamin D, and other vitamin or mineral supplements).

In some embodiments, the active substance is a radiopaque material such as tungsten carbide or barium sulfate.

In certain embodiments, the active substance is one or more specific therapeutic agents. As used herein, the term “therapeutic agent” or also referred to as a “drug” refers to an agent that is administered to a subject to treat a disease, disorder, or other clinically recognized condition, or for prophylactic purposes, and has a clinically significant effect on the body of the subject to treat and/or prevent the disease, disorder, or condition. Listings of examples of known therapeutic agents can be found, for example, in the United States Pharmacopcia (USP), Goodman and Gilman's The Pharmacological Basis of Therapeutics, 10th Ed., McGraw Hill, 2001; Katzung, B. (ed.) Basic and Clinical Pharmacology, McGraw-Hill/Appleton & Lange; 8th edition (Sep. 21, 2000); Physician's Desk Reference (Thomson Publishing), and/or The Merck Manual of Diagnosis and Therapy, 17th ed. (1999), or the 18th ed (2006) following its publication, Mark H. Beers and Robert Berkow (eds.), Merck Publishing Group, or, in the case of animals, The Merck Veterinary Manual, 9th ed., Kahn, C.A. (ed.), Merck Publishing Group, 2005; and “Approved Drug Products with Therapeutic Equivalence and Evaluations,” published by the United States

Food and Drug Administration (F.D.A.) (the “Orange Book”). Examples of drugs approved for human use are listed by the FDA under 21 C.F.R. §§ 330.5, 331 through 361, and 440 through 460, incorporated herein by reference; drugs for veterinary use are listed by the FDA under 21 C.F.R. §§ 500 through 589, incorporated herein by reference.

In certain embodiments, the therapeutic agent is a small molecule. Exemplary classes of therapeutic agents include, but are not limited to, analgesics, anti-analgesics, anti-inflammatory drugs, antipyretics, antidepressants, antiepileptics, antipsychotic agents, neuroprotective agents, anti-proliferatives, such as anti-cancer agents, antihistamines, antimigraine drugs, hormones, prostaglandins, antimicrobials (including antibiotics, antifungals, antivirals, antiparasitics), antimuscarinics, anxioltyics, bacteriostatics, immunosuppressant agents, sedatives, hypnotics, antipsychotics, bronchodilators, anti-asthma drugs, cardiovascular drugs, anesthetics, anti-coagulants, inhibitors of an enzyme, steroidal agents, steroidal or non-steroidal anti-inflammatory agents, corticosteroids, dopaminergics, electrolytes, gastro-intestinal drugs, muscle relaxants, nutritional agents, vitamins, parasympathomimetics, stimulants, anorectics and anti-narcoleptics. Nutraceuticals can also be incorporated into the drug delivery device. These may be vitamins, supplements such as calcium or biotin, or natural ingredients such as plant extracts or phytohormones.

The active substance may be associated with the second polymer and/or present in the plurality of particles in any suitable amount. In some embodiments, the active substance is present in the plurality of particles comprising the second polymer. For instance, the plurality of particle may encompass, entrap, and/or otherwise comprise the active substance. Advantageously, the plurality of particle may comprise the active substance (e.g., the biological material) at relatively high loadings while limiting burst release thereof. In some embodiments, the active substance is present in the plurality of particles in an amount ranging between about 0.01 wt % and about 50 wt %. In some embodiments, the active substance is present in the plurality of particles in an amount of greater than or equal to 0.01 wt %, greater than or equal to 0.05 wt %, greater than or equal to 0.1 wt %, greater than or equal to 0.5 wt %, greater than or equal to 1 wt %, greater than or equal to 2 wt %, greater than or equal to 3 wt %, greater than or equal to 5 wt %, greater than or equal to 10 wt %, greater than or equal to 20 wt %, greater than or equal to 30 wt %, greater than or equal to 40 wt %, greater than or equal to 50 wt %, greater than or equal to 60 wt %, greater than or equal to 70 wt %, greater than or equal to 80 wt %, or greater than or equal to 90 wt %. In certain embodiments, the active substance is present in the plurality of polymers in an amount of less than or equal to about 90 wt %, less than or equal to about 80 wt %, less than or equal to about 70 wt %, less than or equal to about 60 wt %, less than or equal to about 50 wt %, less than or equal to about 40 wt %, less than or equal to about 30 wt %, less than or equal to about 20 wt %, less than or equal to about 10 wt %, less than or equal to about 5 wt %, less than or equal to about 3 wt %, less than or equal to about 2 wt %, less than or equal to about 1 wt %, less than or equal to about 0.5 wt %, less than or equal to about 0.1 wt %, or less than or equal to about 0.05 wt %. Combinations of the above-referenced ranges are possible (e.g., greater than or equal to 0.01 wt % and less than or equal to 90 wt %). Other ranges are also possible.

The biological material may be associated with the second polymer and/or present in the plurality of particles in any suitable amount. In some embodiments, the biological material is present in the plurality of particles in an amount ranging between about 0.01 wt % and about 90wt %. In some embodiments, the biological material is present in the plurality of particles in an amount of greater than or equal to 0.01 wt %, greater than or equal to 0.05 wt %, greater than or equal to 0.1 wt %, greater than or equal to 0.5 wt %, greater than or equal to 1 wt %, greater than or equal to 2 wt %, greater than or equal to 3 wt %, greater than or equal to 5 wt %, greater than or equal to 10 wt %, greater than or equal to 20 wt %, greater than or equal to 30 wt %, greater than or equal to 40 wt %, greater than or equal to 50 wt %, greater than or equal to 60 wt %, greater than or equal to 70 wt %, greater than or equal to 80 wt %, or greater than or equal to 90 wt %. In certain embodiments, the biological material is present in the plurality of polymers in an amount of less than or equal to about 90 wt %, less than or equal to about 80 wt %, less than or equal to about 70 wt %, less than or equal to about 60 wt %, less than or equal to about 50 wt %, less than or equal to about 40 wt %, less than or equal to about 30 wt %, less than or equal to about 20 wt %, less than or equal to about 10 wt %, less than or equal to about 5 wt %, less than or equal to about 3 wt %, less than or equal to about 2 wt %, less than or equal to about 1 wt %, less than or equal to about 0.5 wt %, less than or equal to about 0.1 wt %, or less than or equal to about 0.05 wt %. Combinations of the above-referenced ranges are possible (e.g., greater than or equal to 0.01 wt % and less than or equal to 90 wt %). Other ranges are also possible.

Advantageously, certain embodiments of the composition described herein may permit higher concentrations (weight percent) of active substances such as biologics and/or therapeutic agents to be incorporated as compared to other polymers such as certain conventional hydrogels. In some embodiments, the active substance (e.g., a biological material) may be released from the composition. In certain embodiments, the active substance is released by diffusion out of the composition. In some embodiments, the active substance is released by degradation (e.g., erosion) of the composition (e.g., biodegradation, enzymatic degradation, hydrolysis). In some embodiments, the active substance is released from the composition at a particular rate. Those skilled in the art would understand that the rate of release may be dependent, in some embodiments, on the solubility of the active substance in the medium in which the composition is exposed, such as a physiological fluid associated with tissue surrounding the plurality of particles.

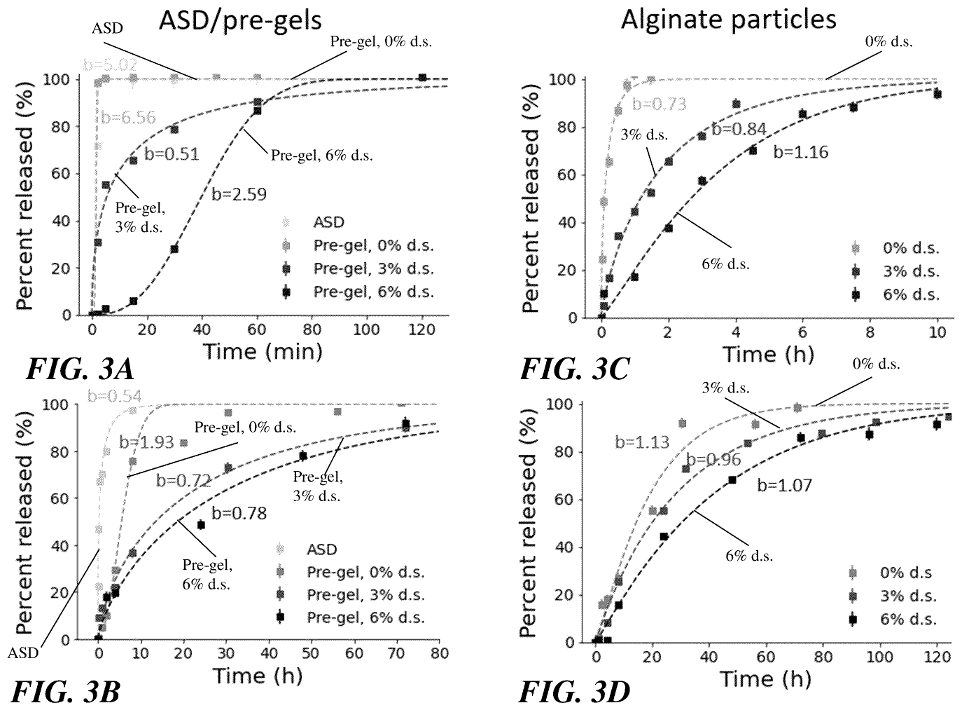

According to some embodiments, the active substance, such as the biological material, may be released from the composition. Advantageously, the active substance may be released in a controlled and/or sustained manner. Unlike conventional hydrogels impregnated with active substances, the composition described herein does not release the active substance in a burst. Rather, the active substance is released over a period of hours, days, weeks, and/or months. An exemplary release profile of the compositions described herein is shown in FIG. 3D.

In some embodiments, between 0.05 wt % to 99 wt % of the active substance initially contained in the composition is released (e.g., in vivo) between 24 hours and 1 year. In some embodiments, between about 0.05 wt % and about 99.0 wt % of the active substance is released (e.g., in vivo) from the composition after a certain amount of time. In some embodiments, at least about 0.05 wt %, at least about 0.1 wt %, at least about 0.5 wt %, at least about 1 wt %, at least about 5 wt %, at least about 10 wt %, at least about 20 wt %, at least about 50 wt %, at least about 75 wt %, at least about 90 wt %, at least about 95 wt %, or at least about 98 wt % of the active substance associated with the composition is released (e.g., in vivo) within about 24 hours, within 36 hours, within 72 hours, within 96 hours, or within 192 hours. In certain embodiments, greater than or equal to 0.05 wt %, greater than or equal to 0.1 wt %, greater than or equal to 0.5 wt %, greater than or equal to 1 wt %, greater than or equal to 5 wt %, greater than or equal to 10 wt %, greater than or equal to 20 wt %, greater than or equal to 50 wt %, greater than or equal to 75 wt %, greater than or equal to 90 wt %, greater than or equal to 95 wt %, or greater than or equal to 98 wt % of the active substance is released (e.g., in vivo) within 1 day, within 5 days, within 30 days, within 60 days, within 120 days, or within 365 days. For example, in some cases, at least about 90 wt % of the active substance associated with the composition is released (e.g., in vivo) within 72 hours.

In some embodiments, the active substance is released from the composition at a particular initial average rate as determined over the first 24 hours of release (the “initial rate”) (e.g., release of the active substance at the desired location internally of the subject, such as an internal cavity). In certain embodiments, the active substance is released at an average rate of greater than or equal to 1%, greater than or equal to 2%, greater than or equal to 5%, least about 10%, greater than or equal to 20%, greater than or equal to 30%, least about 50%, greater than or equal to 75%, greater than or equal to 80%, greater than or equal to 90%, greater than or equal to 95%, or greater than or equal to 98% of the initial average rate over a 24 hour period after the first 24 hours of release. In some embodiments, the active substance is released at an average rate of less than or equal to about 99%, less than or equal to about 98%, less than or equal to about 95%, less than or equal to about 90%, less than or equal to about 80%, less than or equal to about 75%, less than or equal to about 50%, less than or equal to about %, less than or equal to about 30%, less than or equal to about 20%, less than or equal to about 10%, less than or equal to about 5%, or less than or equal to about 2% of the initial average rate over a 24 hour period after the first 24 hours of release. Any and all closed ranges that have endpoints within any of the above referenced ranges are also possible (e.g., between about 1% and about 99%, between about 1% and about 98%, between about 2% and about 95%, between about 10% and about 30%, between about 20% and about 50%, between about 30% and about 80%, between about 50% and about 99%). Other ranges are also possible.

The active substance may be released at an average rate over at least one selected continuous 24 hour period at a rate of between about 1% and about 99% of the initial rate between 48 hours and about 1 year (e.g., between 48 hours and 1 week, between 3 days and 1 month, between 1 week and 1 month, between 1 month and 6 months, between 3 months and 1 year, between 6 months and 2 years) after the initial release.

For example, in some cases, the active substance may be released at a rate of greater than or equal to 1%, greater than or equal to 2%, greater than or equal to 5%, greater than or equal to 7.5%, greater than or equal to 10%, greater than or equal to 15%, greater than or equal to 20%, greater than or equal to 25%, greater than or equal to 30%, greater than or equal to 35%, greater than or equal to 40%, greater than or equal to 50%, greater than or equal to 90%, and/or up to 100% of the initial rate on the second day of release, the third day of release, the fourth day of release, the fifth day of release, the sixth day of release, and/or the seventh day of release.

In certain embodiments, burst release of an active substance from the composition is generally avoided. For example, in some embodiments, at least about 20% of the active substance is released from the composition within 24 hours, between about 40% and about 70% is released during the first day of release (e.g., at the location internally of the subject), and between about 0.05% and about 80% is released during the second day of release. Those skilled in the art would understand that the active substance may be further released in similar amounts during a third day, a fourth day, a fifth day, etc. depending on the properties of the composition and/or the active substance.

The active substance may be released at a relatively constant average rate (e.g., a substantially zero-order average release rate) over a time period of at least about 24 hours. In certain embodiments, the active substance is released at a first-order release rate (e.g., the rate of release of the active substance is generally proportional to the concentration of the active substance) of a time period of at least about 24 hours.

The compositions described herein may have any of variety of suitable properties. In some embodiments, the storage modulus of the composition is relatively high. For example, the storage modulus of the composition may be higher than the storage modulus of the first polymer and/or the storage modulus of the second polymer. Without wishing to be bound by any particular theory, a relatively high storage modulus may be desirable as active substance within the plurality of particles may migrate out in a more sustained manner than active substance in particles having a low storage modulus. In some embodiments, the storage modulus of the composition is at least 2 times, at least 3 times, at least 4 times, at least 5 times, at least 6 times, at least 7 times, at least 8 times, at least 9 times, or at least 10 times greater than the storage modulus of the first polymer and/or the storage modulus of the second polymer. The increase in storage modulus after the composition (e.g., the hydrogel) is formed may be due at least in part to the interactions between hydrophobic domains of the first and the second polymer.

In some embodiments, the composition has a relatively low lower critical solution temperature. In some embodiments, the composition has a critical solution temperature that is close to or equal to body temperature (e.g., 37 degrees Celsius). In some embodiments, the composition has a lower critical solution temperature of less than or equal to 45 degrees Celsius, less than or equal to 40 degrees Celsius, less than or equal to 37 degrees Celsius, less than or equal to 35 degrees Celsius, less than or equal to 30 degrees Celsius, less than or equal to 25 degrees Celsius, and/or less than or equal to 20 degrees. In some embodiments, the composition has a lower critical solution temperature of greater than or equal to 20 degrees Celsius, greater than or equal to 25 degrees Celsius, greater than or equal to 30 degrees Celsius, greater than or equal to 35 degrees Celsius, greater than or equal to 40 degrees Celsius, or greater than or equal to 45 degrees Celsius. Combinations of these ranges are possible (e.g., less than or equal to 50 degrees Celsius and greater than or equal to 20 degrees Celsius). Other ranges are also possible.

In some embodiments, the composition is capable of being administered to a subject. For instance, the composition may be capable of being injected into a subject, such as a mammal (e.g., a human, a mouse, a monkey), using a fluidic delivery device (e.g., a syringe). In some cases, the fluidic delivery device may comprise the composition. In some instances, it may be desirable for the composition to be injectable under application of relatively low forces. For example, in order for healthcare practitioners to administer the composition accurately and in a controlled manner, it may be beneficial for the amount of force needed to dispense the composition from the syringe to be less than or equal to 15 N. A variety of factors may influence the force needed to dispense the composition from a syringe including but not limited to syringe diameter, needle gauge, temperature, pressure, and/or the rheology of the composition. For example, prior to administration, the composition may have a sufficiently low viscosity such that it may be dispensed from a syringe using forces less than or equal to 15 N. In some embodiments, the fluidic delivery device is configured to inject the composition into a subject. For example, the fluidic device may have a relatively large needle gauge and/or a relatively large diameter to allow compositions to be dispensed with limited force. In some embodiments, the viscosity of the composition is less than or equal to 50 cP, less than or equal to 45 cP, less than or equal to 40 cP, less than or equal to 35 cP, less than or equal to 30 cP, less than or equal to 25 cP, less than or equal to 20 cP, less than or equal to 15 cP, less than or equal to 10 cP, or less than or equal to 5 cP. In some embodiments, the viscosity of the composition is greater than or equal to 15 cP, greater than or equal to 20 cP, greater than or equal to 25 cP, greater than or equal to 20 cP, greater than or equal to 25 cP, greater than or equal to 30 cP, greater than or equal to 35 cP, greater than or equal to 40 cP, greater than or equal to 45 cP, greater than or equal to 50 cP. Combinations of these ranges are possible (e.g., less than or equal to 50 cP and greater than or equal to 5 cP). Other ranges are possible.

In some embodiments, the composition comprises a fluid capable of dispersing the first and second polymer (e.g., water). In some embodiments, the composition comprises one or more additives capable of stabilizing the composition for storage. In some cases, the composition may comprise other polymers (beyond the first and second polymer) that serve any of a variety of purposes known in the art and are considered to be included in this disclosure.

The following examples are intended to illustrate certain embodiments of the present invention, but do not exemplify the full scope of the invention.

### EXAMPLE 1

### Injectable Sustained-Release Hydrogel for High-Concentration Antibody Delivery

There is an increasing interest in subcutaneous (SC) delivery as an alternative to the traditional intravenous (IV) for immunotherapies and other advanced therapies. High-concentration formulations of antibodies are generally desired to meet the limited-volume requirements of subcutaneous SC delivery. Despite this need, there remain challenges in delivering stable and injectable antibodies in these high concentrations. Hydrogel encapsulation of amorphous solid antibodies has exhibited desirable stability and injectability of high-concentration antibody formulations. However, the antibody is quickly released from the hydrogel due to the material's porosity, leading to rapid, uncontrolled drug release kinetics undesirable for the drug's efficacy and safety. In this example, a dual-network composite hydrogel is described which leverages interactions between the two polymer networks to exhibit controlled release of the antibody. The solid form of the antibody at high concentrations was loaded within alginate hydrogel microparticles which are then suspended in thermogelling methylcellulose solution to formulate the in situ gelling composite hydrogel. By facile chemical modification of the alginate to tune the microparticles' gel properties and alginate-methylcellulose interactions, the composite system exhibited a delayed release of the drug in a tunable manner and showed a near-zero order release profile for improved therapeutic efficacy. The desirable injectability properties of the composite hydrogel at high antibody concentrations was shown, highlighting the functionalities of dual-network encapsulation. This composite system may be applicable for the sustained delivery of various therapeutic protein forms, such as for high-loading SC formulations.

This example depicts a relatively high concentration, injectable antibody formulation which has a sustained release profile. The formulation consists of antibody ASD-laden alginate microparticles suspended in a methycellulose polymer solution. Upon injection (and hence reaching body temperature), the system may thermally associate in situ to form a composite dual-network system. This associated network may reduce burst release and allow for sustained release of highly concentrated antibody drugs achieved through a simple and gentle formulation process. The case of formulation and desirable flow properties of hydrogel encapsulation in alginate microparticles were integrated with the sustained-release capabilities of thermogelling methylcellulose to form a advantageous dosage form for antibodies.

### Materials

All chemicals used were of analytical grade. Sodium alginate (viscosity 5-40 cP) and methylcellulose (MC, viscosity 15 cP) were purchased from Sigma. Poly(ethylene glycol) (PEG, 3350 kDa) was purchased from Hampton Research. Lyophilized human IgG was purchased from Equitech-Bio, Inc. All other chemicals were purchased from Sigma and used without further purification.

### Composite Hydrogel Formulation Antibody Precipitation

For preparation of amorphous solid dispersions (ASDs) of human total IgG, 500 μL of 40 mg/mL antibody in 50 mM HEPES (N-2-hydroxyethylpiperazine-N-2-ethane sulfonic acid) solution was mixed with 1000 μL of 25% w/v PEG in 50 mM HEPES solution. IgG was precipitated at pH 7.4. Precipitation was carried out in batches at a total volume of 1.5 mL, with each batch yielding 20 mg of the antibody. All solutions were prepared with distilled water and filtered with a 0.2 μm filter. The precipitation mixture was kept at room temperature for 4 hours while rotating at 12 rpm on a tube mixer. Amorphous solid IgG were recovered by centrifugation at 1700 RCF for 30 minutes at 4° C. For later evaluation of the ASDs, the solid antibodies were resuspended in 10% w/v PEG solution buffered with 50 mM HEPES pH 7.4 (storage buffer). ASDs containing MC were prepared by resuspending the solid antibodies in 4% w/v MC solution with 10% w/v PEG and HEPES buffer.

### Alginate Modification

For hydrophobic modification of the alginate polymer, sodium alginate was first oxidized and then further modified by reductive amination of the oxidized alginate. For preparation of the oxidized alginate (OA), sodium alginate was dissolved in DI water at 2% w/v. Sodium periodate was dissolved in DI water at 1.3 mg/mL (for 3 molar % uronic oxidation) and 2.6 mg/mL (for 6 molar % uronic oxidation). 50 mL of the sodium periodate solution was mixed with 100 mL of the sodium alginate solution to carry out the oxidation reaction at room temperature for 24 hours in dark conditions while mixing. After, reaction byproducts and unreacted species were removed from the reaction mixture by dialysis with 3.5 kDa snakeskin dialysis tubes for 48 hours. The product was concentrated using a 5 kDa centrifugal filter and freeze-dried. For preparation of the alkylated alginate, the freeze-dried oxidized alginate was dissolved in phosphate buffer (0.1 M, pH 7) at 2% w/v. Octylamine was added dropwise to the OA solution while stirring, with a molar ratio of octylamine to the oxidized uronic acid units of 5:1. The reducing agent, NaBH₃CN, was dissolved in a small amount of the same phosphate buffer and added to the reaction mixture, with a molar ratio of NaBH₃CN to octylamine of 1:1. NaBH₃CN was used as the reducing agent due to its higher selectivity and reactivity than other reducing agents, particularly at the neutral pH range. The reaction was carried out at room temperature for 48 hours in dark conditions while mixing. After, reaction byproducts and unreacted species were removed by dialysis as described above for 5 days. The final product was concentrated using a 5 kDa centrifugal filter, freeze-dried, and stored at 4° C.

### Antibody Encapsulation

For preparation of the antibody pre-gel suspension, sodium alginate (2% w/v) was dissolved in 10% w/v PEG solution buffered with 50 mM HEPES at pH 7.4. For pre-gels containing MC, methylcellulose was dissolved with the alginate solution at 4% w/v. The resulting solution was filtered using a 0.2 μm filter. The PEG was used to stabilize the antibody precipitates in the amorphous solid state. The alginate solution was added to the solid antibody precipitates in excess and mixed to make a homogeneous suspension, then concentrated via centrifugation at 2500 RCF for 4 hours at 4° C. Excess supernatant was removed and the solid antibodies were resuspended in the remaining solution. To measure the protein concentration, the final pre-gel was diluted 20-fold in phosphate buffered saline (PBS) and measured in a Nanodrop UV-vis spectrophotometer using the 280 nm absorbance method.

For preparation of the antibody-laden particles, the pre-gel suspension (containing alginate) was filled inside a simple microfluidic device made from a plastic syringe barrel connected to a 30 gauge (ID=159 μm, OD=312 μm) blunt-tip needle. The crosslinking bath consisted of 40 mM CaCl₂, 10% w/v PEG, and 50 mM HEPES pH 7.4 and was filled inside a 50 mL centrifuge tube to form the collection bath. The distance from the tip of the needle dispenser to the bath was 3 mm. The device was centrifuged for 15-30 minutes at 400 RCF.

Antibody loading of the final formulations was measured as described above using the 280 nm absorbance method. Encapsulation efficiency of the hydrogel particles was evaluated by measuring the protein concentration in the CaCl₂ cross-linking bath after synthesizing the particles, and comparing to the total amount of antibody used in the pre-gel.

### Rheological Characterization

For characterization of the rheological behavior of methylcellulose and alginate solutions, a stress-controlled rheometer (DHR-3, TA Instruments) was used. An upper-cone geometry (diameter=60 mm, cone angle=1.004°, truncated gap=29 μm) module was used. The solution sample was added to the lower Peltier plate, then the upper cone was lowered to the truncated gap height. To generally minimize surface effects between the sample and the geometry, mineral oil was used to cover the exposed edge of the cone. Water was added to the top of the cone and a solvent trap was used to minimize solvent evaporation from the sample. The sample was conditioned at 20° C. prior to each experiment, including a 60 seconds pre-shear at 10 rad/s and a 60 seconds equilibration. The temperature ramp experiments were performed from 20° C. to 40° C., with a ramp rate of 2° C./min, at a strain amplitude of 1% and a frequency of 1.6 Hz (10 rad/s).

### Swelling Ratio Measurement

Swelling ratio of blank alginate hydrogel particles were measured after cross-linking in a calcium bath. The particles were prepared from a solution of 2% w/v alginate buffered at pH 7.4 with 50 mM HEPES. The cross-linking bath used consisted of 40 mM CaCl₂ and 0.01% w/v Tween 80 surfactant. The particles were synthesized via centrifugal synthesis as described earlier and then rinsed and dried carefully with tissue paper before weighing on an analytical balance. The particles were dried overnight in a vacuum oven and weighed again after drying. The swelling ratio, Qs, was calculated using the following equation:

$${Q}_{s} = \frac{{w}_{s} - {w}_{d}}{{w}_{d}} \qquad ( 1 )$$

where wₛ is the swollen weight of the particles and w_{d} is the dried weight of the particles. All measurements were performed with triplicate samples.

### In Vitro Release Assays

For evaluating release of the antibody from the hydrogel, 50 μL of the hydrogel sample was injected into the bottom of a 2 mL glass vial filled with 1.8 mL of pre-warmed (37° C.) simulated bodily fluid (SBF), which was prepared to mimic the ionic composition of the SC environment with both mono-and divalent ions, as from the literature, with 7.996 g/L sodium chloride, 0.350 g/L sodium bicarbonate, 0.224 g/L potassium chloride, 0.228 g/L potassium phosphate dibasic trihydrate, 0.305 g/L magnesium chloride hexahydrate, 0.278 g/L calcium chloride, 0.071 g/L sodium sulfate, 6.057 g/L tris(hydroxymethyl)aminomethane, and 40 mL/L of 1 M hydrochloric acid. At set time intervals, 400 μL of the supernatant was removed and taken for measurement of protein concentration using the 280 nm UV-vis absorbance method, and the sampled volume was replaced with fresh SBF. Measurements were taken in triplicate.

### Injectability Tests

For evaluating the injectability of the formulations, a ZWICK-ROELL® mechanical testing machine (model BTC-EXMACRO.001) was used. A 500 N full-scale load cell and compression test flat plate attachment were equipped to the machine. A clamp system was used to securely hold the formulation-loaded syringe (plastic, 1 mL, ID=4.78 mm) in place during the test. A 24 gauge (ID=311 μm, OD=566 μm) Luer-lock needle was connected to the syringe. For each displacement-controlled experiment, a stroke distance of 30 mm was used, corresponding to a ˜0.5 mL injection volume. The stroke speed of each experiment was set according to the desired flow rate of injection, and the force exerted to push the syringe plunger down was recorded over the stroke distance of the test. All injectability tests were conducted at ambient conditions. Results and discussion

### Composite Hydrogel Design

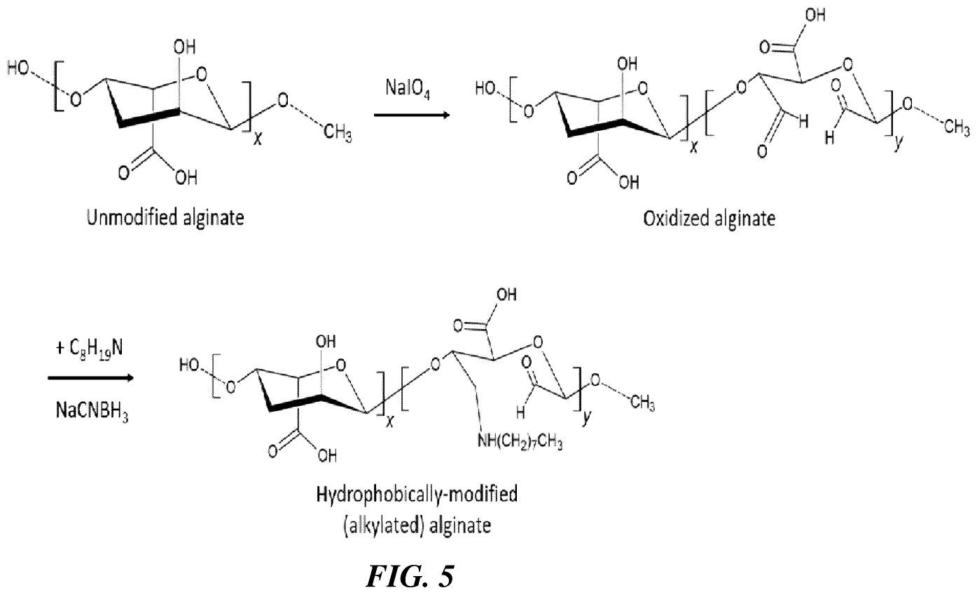

In this example, dual-network antibody-laden composite hydrogels were developed by incorporating ionotropic gelation of alginate to encapsulate highly concentrated antibodies with the thermogelling capability of methylcellulose (MC). As illustrated in FIG. 1, encapsulated amorphous solid immunoglobulin (IgG) antibodies were encapsulated, which are stabilized in the solid state using polyethylene glycol (PEG) into alginate hydrogel microparticles. The IgG ASD-laden microparticles were suspended in a solution of 4% w/v methylcellulose (MC). A second hydrogel network in this composite is formed in situ due to thermal gelation of MC upon injection. Here, human IgG was used as a model antibody drug because the majority of clinically-approved antibodies are IgG types. Interactions between the MC and alginate networks were investigated to tune the thermal gelation of the composite hydrogel towards sustained release of the antibody drug cargo. In addition to the native inter-network interpenetration and hydrogen bonding between alginate and MC, alginate was chemically modified through an oxidation-reductive amination (O-RA) route to graft a hydrophobic side group, octylamine, onto the alginate backbone. Unlike amidation, this method does not consume the polymer's carboxylate groups which may be necessary for alginate cross-linking. Following this route, alginate was first oxidized into a reactive aldehydic intermediate (2,3-dialdehydic alginate) and then underwent subsequent reductive amination, where octylamine was grafted at a degree of substitution of 3 or 6% to induce hydrophobic interactions between the alginate and MC hydrogels. A schematic of the reaction route is shown in FIG. 5. Alginate was modified at relatively low degrees of substitution as oxidation of the polymer at degrees greater than 10% disrupts the backbone structure, resulting in reduction of alginate's cross-linking capability.

Dual-network composite hydrogels, where polymer micro-or nanoparticles are embedded in another polymer matrix, have previously been used to achieve controlled release for protein drug delivery, but have so far been limited to low-concentration formulations (<100 mg/mL). A high-concentration (>100 mg/mL) formulation was presented here which meets dosage requirements for SC administration through a simple, modular formulation approach. Specific polymer-polymer interactions were tuned within the composite hydrogel in order to access a range of drug release kinetics.

### Composite Hydrogel Formulation and Characterization

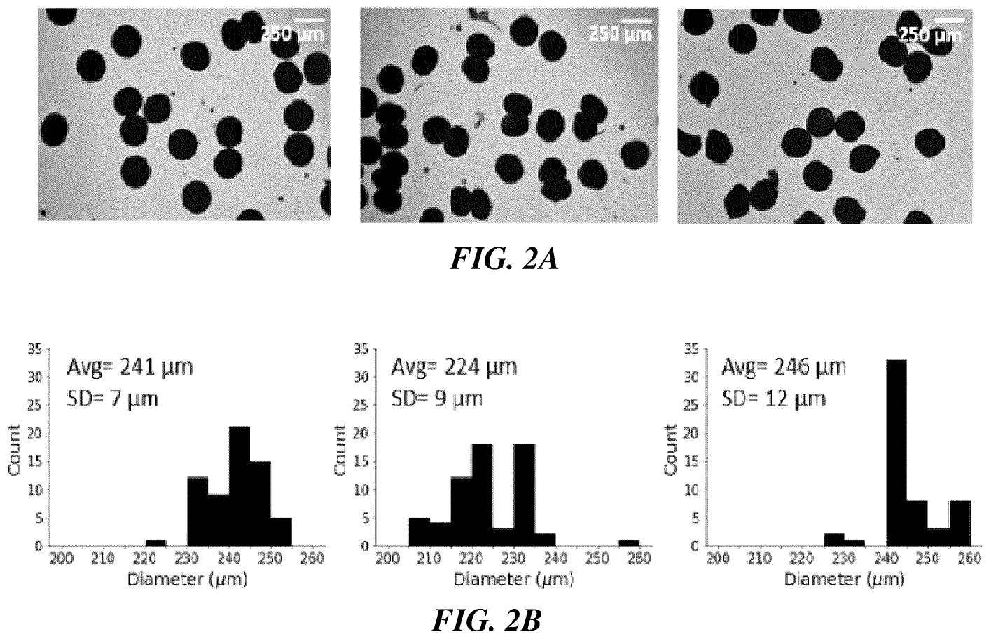

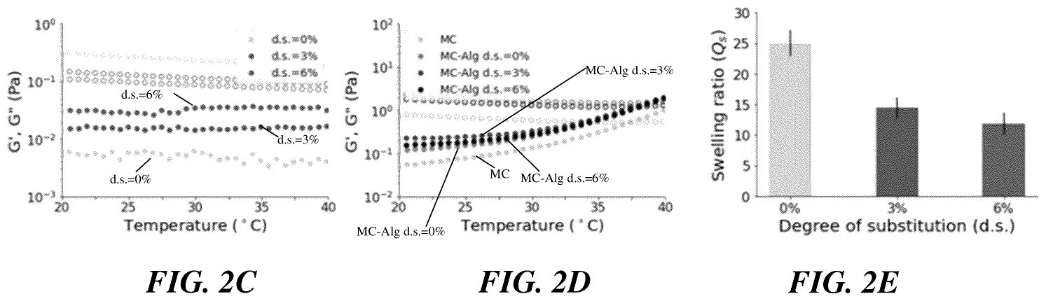

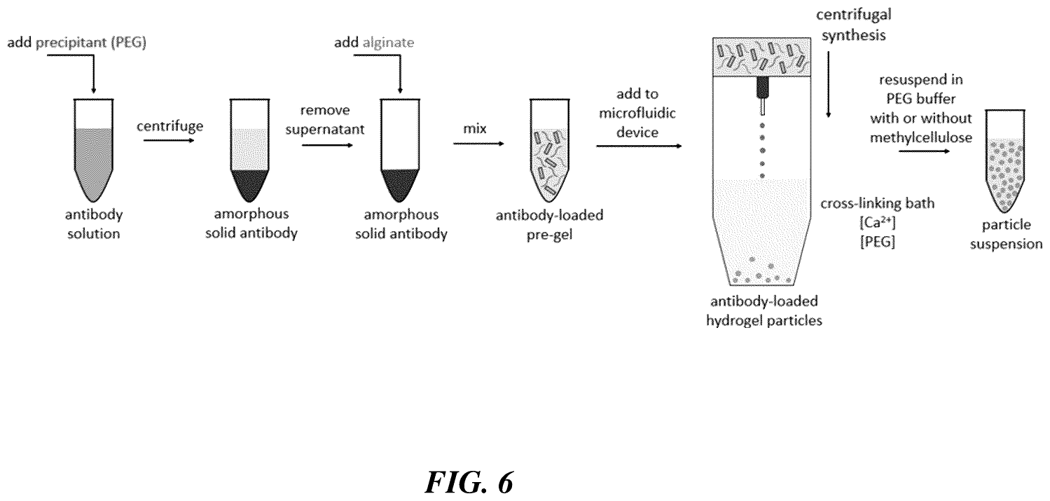

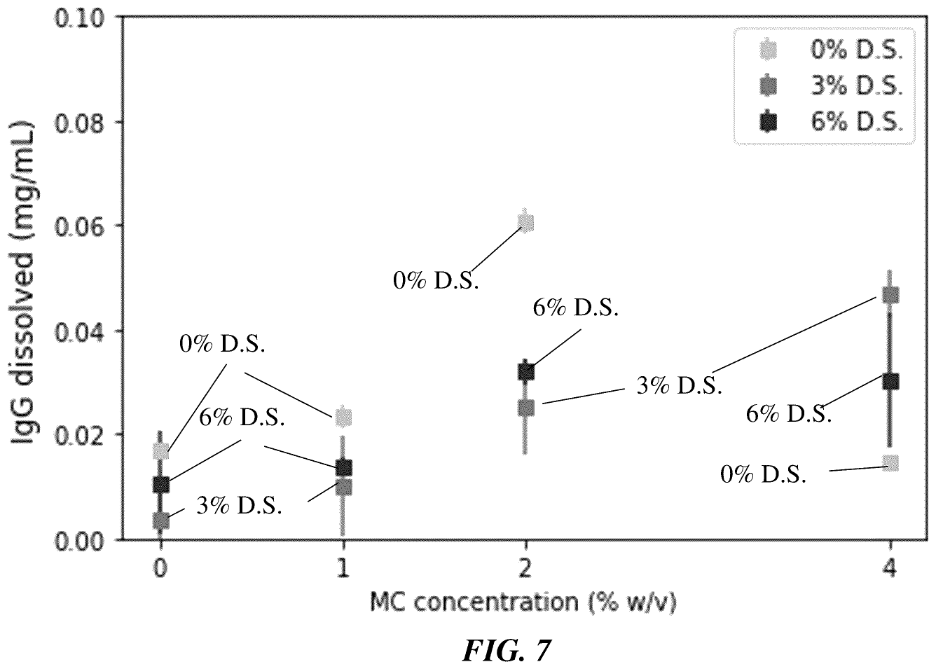

The microparticle formulation process was evaluated for particles synthesized with unmodified (0% d.s.) and modified (3% or 6% d.s.) alginates. Particles with IgG concentration of ˜213 mg/mL were formed, which relates to the final formulation concentration (C_{form}) as C_{form}=(particle loading)*φ where o is the effective particle volume fraction in suspension. The particle loading was measured by determining the volume of the antibody-laden particles and measuring the amount of encapsulated antibody in the particles. A C_{form} of 150 mg/mL was achieved with a particle volume fraction_{φ}=0.70 in the final formulation. The encapsulation efficiency (E.E.) of the particles was defined as the mass of encapsulated antibody over the total mass of antibody in the pre-gel. To measure the encapsulation efficiency, the antibody concentration in the pre-gel and the crosslinking bath were determined. The E.E. for all alginate formulations (Table 1) varied between 98% and <100% w/w which is surprisingly higher than what is typically expected for proteins encapsulated in microspheres (60 to 75%). In FIG. 2A, brightfield microscopy images of the IgG ASD-laden microparticles are shown, synthesized using 0, 3, or 6% substituted alginate (left to right), and the corresponding size distributions are shown below in FIG. 2B. A schematic of the microparticle synthesis process is shown in FIG. 6. The resulting particles are opaque due to the presence of the solid antibodies, which are stabilized with PEG. The particle synthesis process was shown to be robust for modified alginates, indicating that the chemical modification does not affect alginate's ability to encapsulate antibodies, with similar controlled size distributions among all degrees of modification. Modification of the alginate provides an interesting way to modulate the delivery system's properties in a controllable manner, in particular to achieve sustained release. After the alginate microparticles were synthesized, they were suspended in a buffer containing 10% w/v PEG and 4% w/v MC. The antibody remains in its solid form when methylcellulose is present in the buffer with PEG (FIG. 7). In this process, particle synthesis and formation of the composite gel may be independent of each other and thus leads to modular changes to the formulation process. The stability of the released antibody from the hydrogel was not affected in the formulation, either encapsulated in the alginate particle or in the composite hydrogel with methylcellulose, showing no significant change in the monomer percent compared to a control (Table 2).

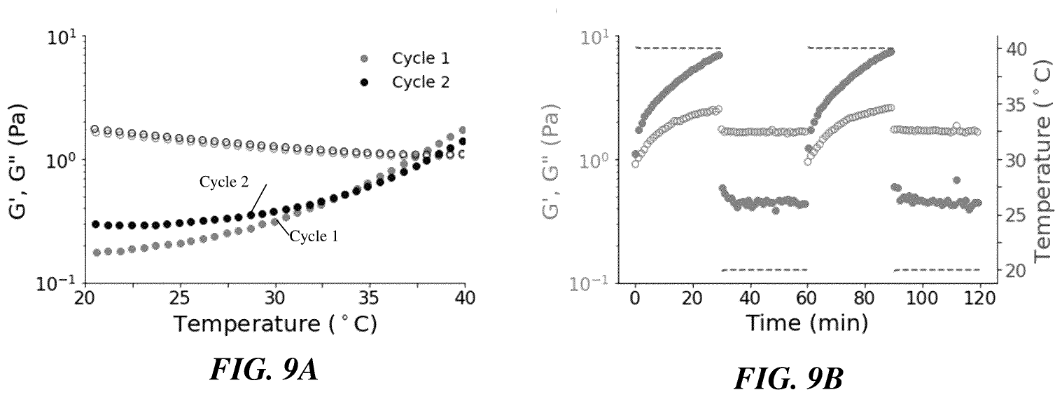

The rheological properties of hydrophobically-modified alginate with varying degrees of substitution were investigated, both alone and with methylcellulose in solution. In FIG. 2C, temperature sweep tests, used to measure the storage modulus (G′) and loss modulus (G″) between 20-40° C., for solutions of 2% w/v alginate polymer are shown. As expected, gelation and thermoresponsive behavior were absent in the unmodified and modified alginates, as G′<G″ over the entire temperature range shown. However, a temperature-dependent increase and decrease was demonstrated in the storage and loss moduli, respectively, for the 3%- and 6%-substituted alginate solutions, which was not observed in the case of the unmodified alginate solution. As hydrophobic interactions increase in strength with temperature, the observed increase in storage modulus (elasticity) with respect to temperature for the hydrophobically-modified is consistent with expectations. There is a significant monotonic increase in G′ with the degree of substitution, resulting in a ˜10-fold greater elasticity at 37° C. for the 6%-substituted alginate compared to the unmodified alginate. This suggests interactions between the side groups of the alginate chains which are contributing to increased viscoelasticity in the polymer solution. FIG. 2D shows temperature sweep tests for solutions of 1% w/v alginate with different degrees of substitution and 4% w/v MC. All solutions showed the formation of a thermo-gel with an apparent gelation temperature (T_{g}), defined as the temperature at which G′>G″, between 37-40° C. Though the composite injectable formulation contains alginate microparticles suspended in a MC-containing buffer, rheometry on MC-alginate solutions was performed to investigate the effect of alginate on the gel structure of MC. The composite hydrogel of alginate and MC may provide improved stability to the formation of the MC thermo-gel. All alginate-MC blends showed a ˜3-fold increase in the gel strength at T_{g} compared to MC alone, and had higher G′ and G″ values across the entire temperature range (FIG. 2D). The presence of alginate may synergistically promote the gelation of MC, leading to the formation of stronger and more thermoresponsive gels. These effects could be explained by hydrogen bonding and entanglement between the two polymers as well as the salting-out effect of the polyanionic alginate which dehydrates the MC network. Hydrophobic interactions between MC and alginate may also be involved. Here, there were not significant differences between the blends with different degrees of alginate hydrophobicity at the tested conditions, which indicates that methylcellulose dominates the gel structure and mechanism. In addition, the thermoreversibility of methylcellulose was not affected by the addition of alginate, as shown in FIGS. 9A-9B. The temperature and time sweeps were performed at a strain amplitude of 1% and frequency of 1.6 Hz.

The swelling ratio (Qₛ) was also measured for blank (no ASD) alginate hydrogel particles synthesized via the centrifugal synthesis process described previously. Briefly, 2% w/v alginate solutions were prepared and passed through the microfluidic device in the centrifuge at 300 RCF. The particles were collected and weighed in their swollen and dried states to determine the swelling ratio, shown in FIG. 2E. The swelling ratio for hydrophobically-modified alginates is significantly lower compared to the unmodified alginate, with a ˜2-fold decrease in Qₛ for the 6%-d.s. alginate. The lower swelling ratios for the modified alginates correspond to the increase in elasticity of the respective polymer solutions, arising from hydrophobic associations between polymer chains. As swelling ratio is typically correlated with the mesh size of hydrogel networks, the decreased Qₛ of the hydrophobically-modified alginate hydrogels indicate a tighter pore structure and slower free diffusion through the hydrogel which is beneficial for sustained release.

### In Vitro Release Studies

In this example, alginate microparticles were used, either unmodified or hydrophobically-modified, composited with MC polymer to form an injectable dual-network system for sustained release of the antibody drug. For an evaluation of the composite hydrogel system, the release profiles for multiple high-concentration solid antibody formulations with the composite system, alginate particles alone, MC hydrogels alone, pre-gels, and the ASD without any hydrogel are shown in FIGS. 3A-3D. The degree of alginate substitution was varied in the pre-gel and particle formulations. Data are shown for technical replicates, n=3, and error bars show standard deviation. All in vitro release assays were performed with formulations with a C_{form} equivalent to 150 mg/mL and with 10% w/v PEG in the initial formulation to stabilize the ASD. The release profiles were fitted to the Weibull equation (Equation 3), an empirical model for drug release kinetics from a hydrogel matrix

$$\frac{{M}_{t}}{{M}_{∞}} = 1 - {e}^{- a {t}^{b}} \qquad ( 3 )$$

where a and b are constants, with b corresponding to the mechanism of drug release. The value of b for each release profile was extracted to quantify the release behavior for different formulations. If b≤0.75, the mechanism is Fickian diffusion, reflecting first-order or burst release kinetics, and if b>1, the mechanism is complex. Values of n between 0.75 and 1 correspond to anomalous transport of a combination between Fickian diffusion and polymer relaxation, which reflects the suppression of burst release and approaches zero-order kinetics as the value of b increases. Details of the model parameters and fits are shown in Table 3.

For the formulations without methylcellulose (FIG. 3A and FIG. 3C), release of the antibody was achieved within minutes or hours. The ASD (solid antibody without hydrogel) and pre-gel (solid antibody with uncross-linked alginate) containing unmodified (0% d.s.) alginate saw burst release within the first few minutes of the release test (FIG. 3A). In these cases, the extremely rapid drug release led to practically asymptotic profiles with unphysical values of the Weibull exponent (b>5). The hydrogel particles containing unmodified (0%-d.s.) alginate also saw a burst release effect (FIG. 3C). The formulations with hydrophobically-modified alginate had pronounced differences in the release profiles, even without MC. For the pre-gels, burst release was suppressed as the alginate's degree of substitution increased. The increasing hydrophobicity of the modified alginates contributes to slower water diffusion through the pre-gel and thus a slower dissolution of the solid antibody. The same observation was made in the cross-linked alginate particles (FIG. 3C), where 100% of the total antibody was released from the unmodified particles within 1 hour, while only 44.7% and 17.2% of the antibody was released from the 3%-d.s. and 6%-d.s. particles in the same time, respectively. The release mechanism from the particles changed from first-order, diffusion-dominated kinetics for the unmodified particles (b=0.73) to erosion-controlled transport for the particles with 6%-d.s. alginate (b=1.16). The difference in release kinetics may be due to the reduced swelling behavior which was observed in the modified alginate particles, as well as interactions between IgG molecules and the hydrophobic side groups slowing transport of IgG.

FIGS. 3B and 3D show the release profiles for the formulations with 4% w/v MC, either blended into the ASD or pre-gel (FIG. 3B) or in the suspension surrounding the hydrogel particles (FIG. 3D). In the case of the MC-containing formulations, release is achieved on the order of hours to days, a ˜10-fold increase in time scale compared to the formulations without MC. The difference in release time scale may be due to formation of the thermo-gel depot upon injection into the release medium. Burst release was generally suppressed in these MC-containing formulations, with the pre-gels showing 5-13% and the particles showing <1-2% release of the total antibody within 1 hour. The ASDs and pre-gels showed primarily first-order release with b=0.54-0.78 (FIG. 3B), indicating that diffusion through MC controls the release mechanism in this case, except for the 0% modified pre-gel, which has a sigmoidal release profile similar to those in FIG. 3A. When the alginate is cross-linked into particles (FIG. 3D), burst release is further suppressed and the release kinetics are in the range of erosion-controlled release (b=0.96-1.13). The difference in the dominant release mechanism between the pre-gels and hydrogel particles may indicate that the cross-linking of alginate contributes to slower diffusion and more linear release over time in the composite system. The presence of the alginate network, in conjunction with MC, plays an valuable role in achieving sustained release without an initial burst. Additionally, inter-network penetration between the MC and alginate networks could result in adsorption of MC onto the particle, leading to slower diffusion of the drug through the pores of the alginate particle. In the case of the hydrophobically-modified alginates, interactions between the networks are enhanced, coupled with the lower water permeability and polymer-drug interactions of the hydrophobic alginate particles. These effects result in increasingly sustained release with the degree of alginate substitution, where the release over a few days (t₈₀=2.6 days for 6%-d.s. particles in MC) was observed. This time scale of release indicates that the composite hydrogel system may be effective for short-lived antibodies (t_{1/2}<7 days), helping to extend the effective duration of the dosage and reduce the maximum serum concentration which is especially desirable for high-dose formulations.

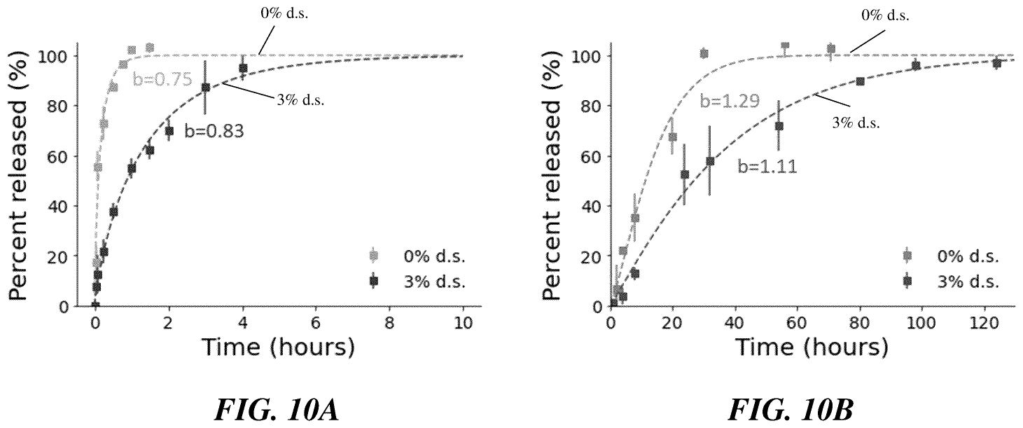

Compared to the alginate particles or MC hydrogel alone, the composite hydrogel showed sustained release, reduced burst release, and more erosion-controlled (zero-order) kinetics, which are desired features for drug delivery systems. To demonstrate that the control over release kinetics is consistent across several independent samples, in vitro release tests were replicated in multiple parallel samples (n=3) for select formulations, which are shown in FIGS. 10A-10B. Data are shown for multiple sample replicates, n=3, and error bars show standard deviation. The simultaneous high-loading and sustained-release capacities of the composite system is a unique feature, as typical hydrogels only achieve loadings of 0.01-1 mg/mL for biological molecules, and other ‘high-loading’ formulations do not exceed >100 mg/mL in antibody concentration. Another advantage of this composite system is the case of formulation by which diverse release profiles can be obtained, as the particles and the surrounding suspension media can be manipulated separately then blended together to yield the final formulation. For all formulations, complete or nearly complete (90-100%) release of antibody from the hydrogel was reached. For the particles where 100% release was not reached over the duration of the test, some amount of antibody could be entrapped within low-porosity regions of the hydrogel.

### Injectability Studies

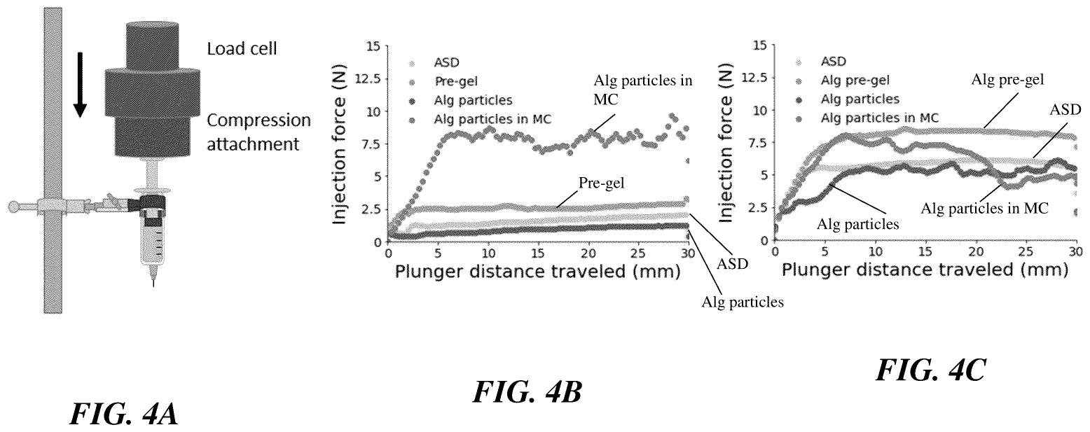

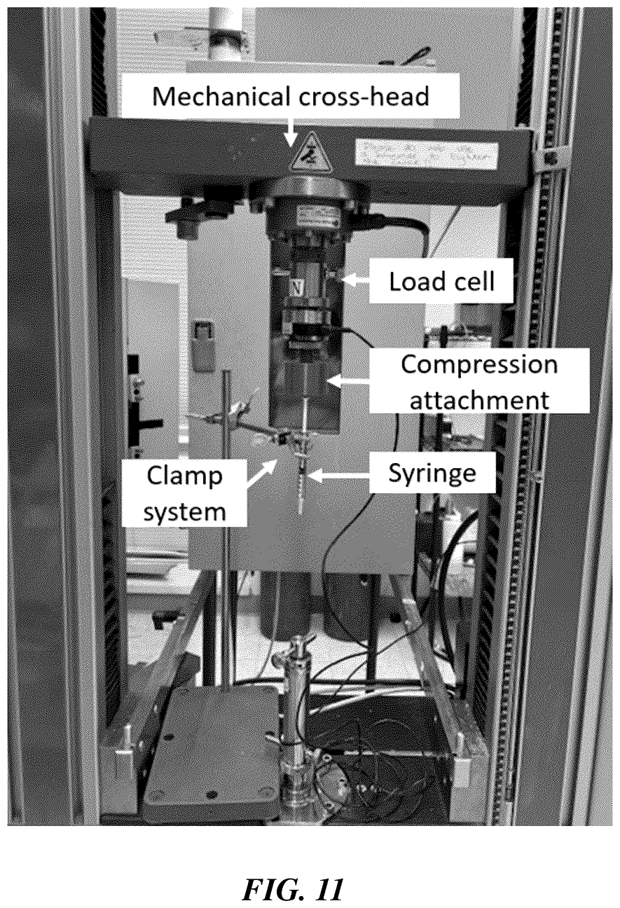

To assess injectability of the formulations, injection force tests were performed. Although material properties of the formulation such as viscosity and storage and loss modulus are relevant, they do not correlate directly to injectability for non-Newtonian solutions. Injection force is a clinically relevant measurement and the test yields quantitative results in a relatively simple manner. A schematic for the injection force testing set-up is shown in FIG. 4A. The test was performed using a ZWICK-ROELL mechanical testing machine, equipped with a 500-N load cell and a custom 3D-printed attachment for compression of the syringe plunger. The hydrogel formulation was loaded into the syringe and an even downward force was applied onto the syringe plunger. An image of the injectability testing set-up is shown in FIG. 11.

FIGS. 4B-4C show the injection force over the distance which was traveled by the plunger during the test at an injection rate of 25 μL/s ('slow' injection) and 150 μL/s ('fast' injection), respectively, for various formulation configurations. A higher injection rate is desirable for reducing the total duration of injection, but previous studies have found higher flow rates to be more painful, due to an increase in back pressure under the skin. Therefore, these two injection rates were chosen to evaluate injectability at clinically relevant limits. All formulations tested had a C_{form} of 150 mg/mL. The tests were performed using a 24-gauge needle, which is within the range of needle bore sizes for subcutaneous injection. The injection force for formulations with hydrogel particles are compared to the ASD and pre-gel formulations, and the formulation with particles were synthesized with unmodified alginate to provide a baseline for performance.

All the formulations tested had a maximum injection force (Fₘₐₓ) less than 10 N, which is well below the recommended acceptable maximum injection force for clinical use (20 N). As observed in FIGS. 4A-4C, each tested formulation experiences a ‘start up’ time at the beginning of the test where the injection force monotonically increases before reaching a plateau, at which the injection force can be averaged to yield the mean injection force (F̅). It was noted here that the injection force profiles experience some variations across the distance travelled by the plunger, suggesting there are local differences in the distribution of the samples. These variations are especially pronounced in the case of the alginate particles (either with or without methylcellulose), likely due to the reversible build-up and breakage of weak local structures as pressure is applied to the syringe plunger. At both tested flow rates, the pre-gels had a higher F̅ than the ASDs alone, due to the alginate which makes the pre-gel more viscous. Notably, the pre-gels also had a higher F than the particles suspended only in 10% w/v PEG. Without wishing to be bound by any particular theory, antibody-loaded hydrogel particles may have desirable flow behavior due to the spherical shape minimizing the surface area exposed for protein interactions, as well as the particles being soft and deformable even at high volume fractions. The results here show that particle formulations have better injectability than equivalently-formulated pre-gels.

At the slow flow rate, the formulation for antibody-loaded particles suspended in 4% w/v methylcellulose had a significantly higher F̅ than the other formulations, due to the viscosity of the methylcellulose (FIG. 4B). However, this difference in injectability is not observed at the higher flow rate. Theoretically, the Hagen-Poiseuille equation predicts that injection force should scale proportionally with the volumetric flow rate. However, all formulations showed a less-than-proportional increase in injection force with the flow rate due to shear-thinning properties of the formulations. Particles suspended in 4% methylcellulose had a lower F at the high flow rate, indicating significant shear-thinning behavior due to the methylcellulose (FIG. 4C), although Fₘₐₓ is similar between the two flow rates. As discussed, the particles suspended in methylcellulose experience structural heterogeneities which result in ‘bumpy’ injection force profiles at both flow rates. At the slow flow rate, the longer residence time for the particles in the syringe could also contribute to the buildup of heterogeneous structures which explain the higher F compared to the fast flow rate. Overall, it was shown that the hydrogel particles maintain acceptable injectability properties, even when suspended in viscous polymer solutions.

### Overall Composite Hydrogel Formulation Scheme

FIG. 6 shows the overall formulation process for the composite hydrogel. First, amorphous solid forms of the antibody were prepared via precipitation (recovery=99.4%±0.1). Next, a sodium alginate solution (2% w/v) mixed into the solid antibodies to form the pre-gel, which was then passed through a microfluidic device for centrifugal synthesis. In this device, the pre-gel is extruded through a needle by centrifugal force, forming droplets which are collected in a cross-linking bath containing calcium chloride (CaCl₂), allowing the alginate to ionically cross-link into hydrogel particles. PEG (10% w/v) is present in both the pre-gel and calcium bath to prevent dissolution of the solid antibodies. Finally, to form the composite in situ gelling hydrogel, the particles were suspended in a buffer containing 10% w/v PEG and 4% w/v MC. The final dosage form was be prepared using only three gentle steps (precipitation, encapsulation, and suspension), without the use of chemical reactions or an organic phase.

Encapsulation efficiencies for alginate microparticles with different degrees of hydrophobic substitution were measured and are shown below in Table 1.

TABLE 1
Encapsulation efficiency of IgG ASD-laden alginate microparticles.
Alginate degree of substitution

| 0% | 3% | 6% |
|---|---|---|
| 99.6% ± 0.1 | 98.3% ± 0.3 | 99.9% ± 0.1 |

### Solubility of IgG ASD in Methylcellulose

For evaluating the solubility of the amorphous solid antibody in formulations containing methylcellulose to ensure the stability of the solid phase in the composite hydrogels, IgG ASD-laden hydrogel particles were prepared as described earlier. The samples were transferred to a microcentrifuge tube and excess storage buffer was removed to adjust the total IgG content in each tube to 3 mg. 300 μL of storage buffer with different w/v % concentrations of MC were added each tube and the samples were left to equilibrate with the buffer at room temperature (˜22° C.). After 24 h, the protein concentration in the supernatant was measured using the 280 nm absorbance method.

As in FIG. 7, the solubility of IgG was observed to be relatively low (0.1 mg/mL) for all formulations.

The solubility of the ASDs are not significantly affected by the presence of methylcellulose in the storage buffer, which indicates that the majority of IgG (>99%) in the formulations remains in its solid form, and thus encapsulated in the particles. There is no substantial ‘leakage’ of IgG from the particles when stored in 4% w/v methylcellulose solution and the solid form of the antibody is maintained.

### Size Exclusion Chromatography (SEC) of Released IgG

Analytical SEC was used to determine the quantity of antibody monomer and aggregates from IgG ASD-laden alginate particles. For this purpose, an AKTA FPLC instrument (GE HEALTHCARE) was used, with a Superdex 200 Increase 10/300 GL analytical SEC column. SEC experiments were carried out at a flow rate of 0.5 mL/min in a phosphate buffered saline (PBS) at pH 7.4. Select conditions were used to characterize the quality of the released IgG antibody in different formulations. For the control experiment, lyophilized IgG powder as received was dissolved into PBS and analyzed. The SEC results are tabulated below in Table 2. Experiments were performed in triplicate (n=3) for each condition, with standard deviations reported.

TABLE 2
Stability of amorphous IgG evaluated
using size exclusion chromatography.

| | Monomer % | Aggregates % |
|---|---|---|
| Control | 81.6 ± 1.0 | 18.4 ± 1.0 |
| Released from alginate particles, 0% d.s. | 84.9 ± 1.5 | 15.1 ± 1.5 |
| Released from alginate particles, 6% d.s. | 82.8 ± 0.9 | 17.2 ± 0.9 |
| Released from alginate particles in | 80.2 ± 1.3 | 19.8 ± 1.3 |
| methylcellulose, 0% d.s. | | |
| Released from alginate particles in | 80.7 ± 0.7 | 19.3 ± 0.7 |
| methylcellulose, 6% d.s. | | |

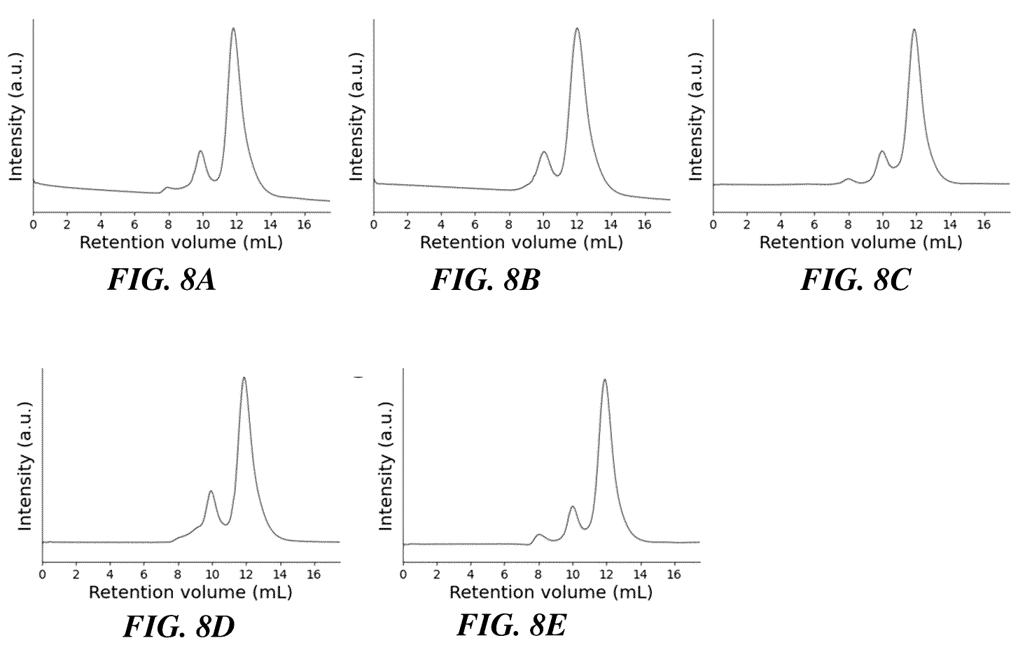

As seen in Table 2, the quality of the IgG released from alginate particles is not significantly different from the control (>80% monomer), indicating that IgG remains stable when formulated into the hydrogels, both for alginate alone and in the composite alginate-methylcellulose hydrogel. In addition, the degree of alginate modification does not affect the stability, with 0% d.s. and 6% d.s. alginate hydrogels showing similar monomer compositions across conditions. Characteristic UV traces for each condition are available in FIGS. 8A-8E.

### Thermoreversibility of MC-Alginate Mixtures

To demonstrate the thermoreversibility of methylcellulose and alginate composites, the rheological behavior of a MC-alginate solution was characterized using multiple temperature sweep cycles. A temperature ramp was performed on the sample from 20° C. to 40° C. at a rate of 2° C./min. Between each ramp cycle, the sample was cooled to 20° C. and equilibrated for 15 minutes before repeating the same ramp. The results of both cycles are shown below in FIG. 9A. The sample has similar temperature sweep profiles in each run with some small hysteresis.

Temperature jump experiments were also performed to compliment the temperature ramp data. For thermally gelling soft matter, the evolution of structure (and hence moduli) in a temperature ramp versus temperature jump experiment may differ. Data for temperature jump experiments are show in FIG. 9B. The temperature was stepped between 20° C. and 40° C. for 2 cycles, and each step had a duration τ=30 minutes.

The sol-gel transition of the MC-alginate mixture is reproducible upon cooling, showing the presence of alginate in the solution does not affect methylcellulose's native thermoreversibility. In addition, the solgel transition temperatures are not affected by multiple heating/cooling cycles. When gradually heated from 20° C., the MC-alginate solution shows a consistent apparent gelation temperature of ˜37-38° C. (FIG. 9A). Similarly, as shown in FIG. 9B, G′>G″ at 40° C. while G″<G′ at 20° C. in both cycles, demonstrating that the sol-gel transition is reversible when alginate is mixed with methylcellulose. The transition from a gel to sol and then sol to gel is quick during temperature jumps. Furthermore, temporal evolution of both the G′ and G″ curves in the repeated cycles are very similar. The MC-alginate mixture is in solution at room temperature and becomes a semi-solid gel at physiological temperatures across multiple cycles, indicating its reversibility and suitability for pharmaceutical applications. The storage modulus was observed to increase over time when the sample is held at 40° C., indicating the formation of a stronger gel over time which may lend to the release behavior of the in situ gel as discussed.

### Drug Release Model Fitting

To fit the release profiles presented in this work, the Weibull model was used. Though the Peppas (power-law) model (Equation 1) is can be applied for drug release from hydrogel-based delivery systems, it is a short-time approximation (valid for M/M≤0.60). Thus, the Peppas model does not provide a good fit across the entire release profile.

$$\frac{{M}_{t}}{{M}_{∞}} = {kt}^{n} \qquad ( Equation 1 )$$

However, the value of the model exponent (n) in the Peppas model can provide a better physical and kinetic basis than the Weibull model. The interpretation of n is similar to that of b in the Weibull model, such that n≤0.43 indicates a diffusion-controlled, first-order release mechanism, n>0.85 indicates a polymer erosion-controlled, zero-order release profile, and values of n between 0.43 and 0.85 indicate anomalous transport in between the two limits. For the purposes of comparison, the exponent values for both the Weibull and Peppas model and their fit (R²) across the entire range of release data are shown in Table 3. As shown in Table 3, the value of b generally corresponds with the value of n, suggesting good agreement of the release mechanism regimes and a valid basis for the Weibull model used in the main work.

TABLE 3
Drug release model parameters and fits for the Weibull and Peppas models.

| | Formulations without methylcellulose | | | | Formulations with methylcellulose | | | |
|---|---|---|---|---|---|---|---|---|
| | Weibull model | | Peppas model | | Weibull model | | Peppas model | |
| | b | R2 | n | R2 | b | R2 | n | R2 |
| ASD only | 5.02 | 0.999 | n/a* | | 0.54 | 0.981 | 0.30 | 0.737 |
| Pre-gel, 0% d.s. | 6.56 | 0.999 | n/a* | | 1.93 | 0.988 | 0.56 | 0.844 |
| Pre-gel, 3% d.s. | 0.51 | 0.988 | 0.29 | 0.946 | 0.722 | 0.991 | 0.52 | 0.878 |
| Pre-gel, 6% d.s | 2.59 | 0.999 | 1.70 | 0.758 | 0.779 | 0.979 | 0.47 | 0.969 |
| Particles, 0% d.s. | 0.727 | 0.990 | 0.40 | 0.978 | 1.13 | 0.978 | 0.79 | 0.861 |
| Particles, 3% d.s. | 0.838 | 0.990 | 0.56 | 0.986 | 0.962 | 0.992 | 0.66 | 0.850 |
| Particles, 6% d.s. | 1.16 | 0.991 | 0.76 | 0.938 | 1.07 | 0.995 | 0.76 | 0.866 |

*Peppas model was not valid here as all data points in this profile were Mₜ/M_{∞} > 0.60

### Multiple Sample Replicates of in Vitro Release Tests

Select alginate particle formulations, with and without methylcellulose, were chosen to demonstrate consistent control in in vitro release kinetics across multiple samples of the same formulation conditions, whereas the data described else wherein the present disclosure (FIGS. 3A-3D) is taken as technical replicates. In vitro release tests were performed for multiple samples (n=3) of select formulation conditions. The results are shown below in FIGS. 10A-10B.

In general, consistent release kinetics were found across different samples in each of the formulation conditions and show that trends in release behavior remain the same, as according to the value of b in the Weibull model fits. The b values for formulations without methylcellulose can be controlled by the degree of alginate substitution, going from the diffusion-controlled release regime (b≤0.75) in the case of the unmodified alginate particles to approaching the erosion-controlled regime as the value of b increases with degree of alginate substitution. For the formulations with methylcellulose, all profiles again show release behavior within the erosion-controlled release regime (b>1) regardless of the degree of alginate substitution. The individual Weibull and Peppas fits for each of the replicated samples are reported below in Table 4.

TABLE 4
Drug release model parameters and fits for the Weibull and Peppas models.

| | Formulations without methylcellulose | | | | Formulations with methylcellulose | | | |
|---|---|---|---|---|---|---|---|---|
| | Weibull model | | Peppas model | | Weibull model | | Peppas model | |
| | b | R2 | n | R2 | b | R2 | n | R2 |
| Particles, 0% d.s. | 0.727 | 0.990 | 0.40 | 0.978 | 1.13 | 0.978 | 0.79 | 0.861 |
| | 0.782 | 0.936 | 0.53 | 0.795 | 1.34 | 0.967 | 0.80 | 0.886 |
| | 0.795 | 0.950 | 0.55 | 0.771 | 1.45 | 0.996 | 0.90 | 0.720 |
| Particles, 3% d.s. | 0.838 | 0.990 | 0.56 | 0.986 | 0.962 | 0.992 | 0.66 | 0.850 |
| | 1.01 | 0.982 | 0.49 | 0.913 | 1.07 | 0.949 | 0.72 | 0.877 |
| | 0.875 | 0.931 | 0.49 | 0.961 | 1.15 | 0.988 | 0.79 | 0.940 |

The formulation of injectable composite hydrogels consisting of alginate microparticles and thermoresponsive methylcellulose hydrogel was described for the delivery of high-concentration antibodies. The formulation process can be modular as the synthesis of the microparticles and the composite hydrogel can be accomplished independently from each other. The alginate was modified with hydrophobic side groups to tune the release behavior of the particles, and alginate particles were prepared by gentle ionic cross-linking via centrifugal synthesis. Synergistic improvement of methylcellulose's thermoresponsive behavior with the addition of alginate was shown, and in vitro release studies demonstrated that the composite system suppresses burst release effect and sustains release of a model antibody drug, IgG, compared to the particles or methylcellulose hydrogel alone. The in vitro release profiles were fit to the Weibull model, where the model parameter b was used to characterize the kinetics. A wide range of release kinetics (b=0.73-1.16) was demonstrated for formulations with alginate microparticles with the ability to tune release based on the degree of alginate modification and the methylcellulose content. The composite system also showed desirable injectability properties at clinically relevant testing conditions. Overall, the results suggest that the dual-network hydrogel composite system is a advantageous route to provide sustained-and controlled-release delivery of highly concentrated antibodies.

The composite hydrogel may be used as an injectable depot-forming drug delivery system for controlling the release behavior of antibodies in a tunable manner. The composite system also maintains the advantages of hydrogels in general for encapsulation and delivery of therapeutics, including its biocompatibility and stabilization of the antibody cargo in its solid form. In addition, the hydrogel's softness, deformability, and shear-thinning behavior enable case of injection for highly concentrated dosage forms. Though this approach was demonstrated for formulating high-concentration amorphous solid antibodies, it may also be a suitable concept for other physical states of the antibody, including crystalline solids and coacervates. Though IgG was used as a model drug in this example, given that the encapsulation approach is not specific to the therapeutic molecule and only relies the ability of the molecule to remain in a solid form, it is possible to expand this system to be a viable formulation platform for any therapeutic molecule in general, including small molecules, monoclonal antibodies, peptides, nucleic acids, and advanced biologics. Moreover, different crosslinking chemistries can be incorporated in either the microparticle (i.e. Michael-type addition with functionalized alginate) or the thermo-gelling matrix (i.e. citric acid small molecule linker for methylcellulose hydrogels).

### EXAMPLE 2

### Injectable Thermally Gelling Hydrogel Composites for Controlled Therapeutic Delivery

This example addresses the demand for improved compositions for controlled or sustained drug delivery. The example discloses an injectable hydrogel of a polymer which thermally gels via hydrophobic association composited with orthogonally-crosslinked hydrogel microparticles. The hydrogel particles encapsulate an active pharmaceutical ingredient (API) and are made from a polymer with hydrophobic groups which synergistically associate with the first polymer and contribute to its gelation. The purpose of this example is to provide a surprising modality for controlled delivery of an active pharmaceutical ingredient. Specifically, the hydrophobic associations within the hydrogel composite act as a lever with which to tune release of API from the hydrogel particle. The technology solves the problem of rapid initial release rates and uncontrolled release kinetics common to hydrogel-based drug delivery systems. Fast and uncontrolled delivery of API results in the need for frequent dosing of the drug and potential toxicities in vivo. Advantages of the invention include case of chemical modification, versatility in methods of preparation and compositions, and tunability of release kinetics according to diverse target pharmacokinetic profiles and dosing regimens.

Thermally gelling polymers which undergo a phase transition from liquid at room temperature to gel at body temperature have wide therapeutic applications as in situ-forming gels. Separately, thermally gelling matrices and hydrogel micro-or nanoparticles made from thermally gelling polymers have been investigated for sustained-release delivery but still suffer from an initial ‘burst release’ or concentration-dependent (first-order) kinetics. Hydrogel particles have also been embedded within a thermally gelling matrix for controlled-release applications, but previous formulations have been limited to low drug loadings (<1-50 mg/mL) and do not take advantage of tunable hydrophobic associations between the particles and the thermal gel. Thus, the development of improved injectable formulations for controlled, sustained delivery of drug molecules is desired.

The present example provides a desirable composition for controlled delivery of therapeutic molecules, addressing the limitations of current hydrogel-based drug delivery systems. Specifically, the purpose of the invention is to achieve tunable, sustained (order of days to weeks) drug release at a zero-order (or near-zero-order) rate from an injectable hydrogel formulation. Unlike first-order kinetics and ‘burst release’ effects, zero-order kinetics ensures a constant rate of drug release over time, which is desirable for enhancing therapeutic efficacy while minimizing adverse effects and inconsistent pharmacokinetic profiles. Furthermore, the tunability of the composition enables long-acting, safe therapies whereby the composition may be designed according to the desired pharmacokinetic profiles and clinical needs.

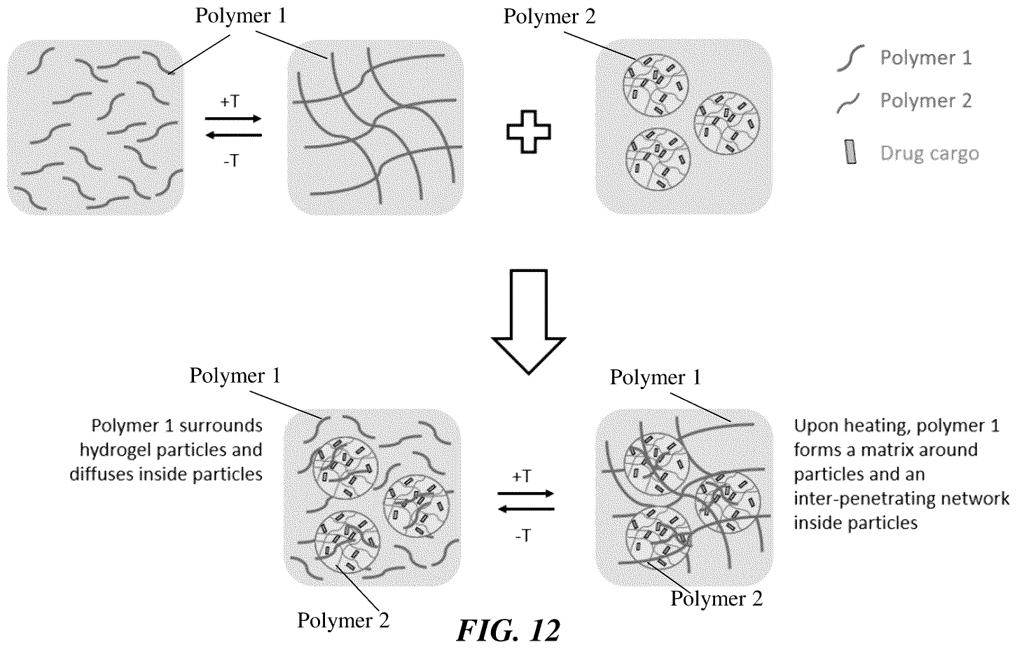

An injectable hydrogel composite of a polymer which thermally gels via hydrophobic association (polymer 1) at body temperature and orthogonally-crosslinked hydrogel particles suspended within the thermal gel are generally described. The hydrogel particles encapsulate an active pharmaceutical ingredient (API) and are formed from a polymer with hydrophobic functional groups (polymer 2) which associate with functional groups on polymer 1 and contribute to its thermal gelation. In some embodiments, chains of polymer 1 are able to diffuse into crosslinked polymer 2 network, forming an additional barrier to drug release upon injection into the body. The interactions between polymers 1 and 2 lead to a synergistic effect on the properties of the hydrogel composite and release of the API. The hydrogel particles from polymer 2 may be either prepared in the presence of polymer 1 or composited with polymer 1 after preparation. A schematic depiction of the hydrogel composite is shown below in FIG. 1 and FIG. 12.

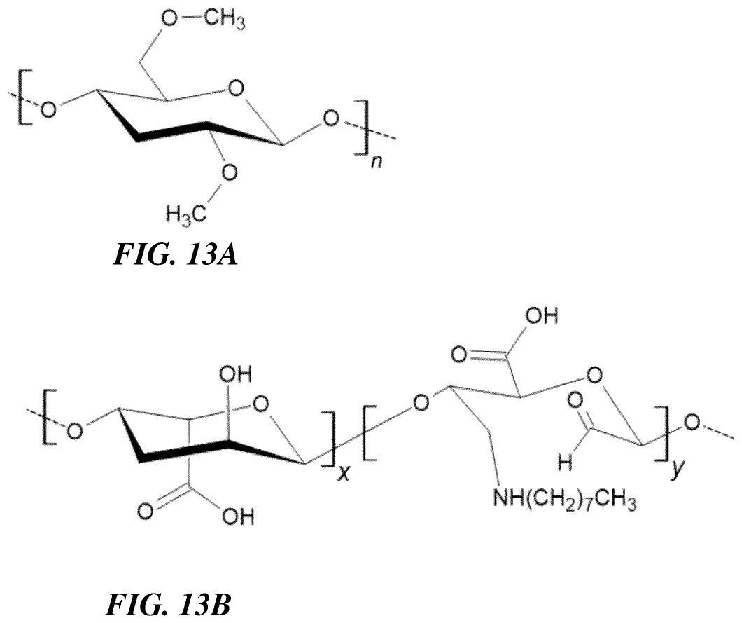

In one embodiment, polymer 1 is methylcellulose (substituted 27.5-31.5%) with a molecular weight of 14 kDa and polymer 2 is alkylated alginate (substitution 3%) with a molecular weight of <75 kDa and a mannuronic-to-glucuronic acid ratio of >1:1. Methylcellulose is a methyoxy-substitute derivative of cellulose which shows LCST (lower critical solution temperature) behavior around physiological temperatures (37° C.). Alginate is a polysaccharide with carboxylic acid groups that can ionically bind with cations (i.e. calcium) to form a crosslinked network. In this embodiment, the alginate was partially oxidized at its C2-C3 position and a long-chain alkyl group was attached to the resulting oxidized residues. The chemical structures of both polymers (post-modification) are displayed in FIGS. 13A-13B below.

In this embodiment, the alginate particles, crosslinked with calcium ions, encapsulate a solid form of a therapeutic protein (human IgG) at high drug loadings (>100-200 mg/mL) and are suspended in methylcellulose sol at a particle volume fraction in the range of 0.5-0.8. The resulting embodiment is an injectable suspension of alginate microparticles (having an average largest cross-sectional dimension between 100-500 microns) in a methylcellulose polymer network. In Appendix C, drug release profiles are shown, generated in vitro, for one variation of this embodiment in which the alginate particles are formulated at 2% weight-by-volume (w/v) polymer and the methylcellulose sol at 4% w/v polymer.

In other embodiments, different thermo-gelling polymers were considered for polymer 1 (including, but not limited to, hydroxypropylmethylcellulose, carboxymethylcellulose, chitosan, poly(N-isopropylacrylamide), and poloxamers) and different cross-linkable polymers for polymer 2 (including, but not limited to, polyethylene glycol or other polyalkene oxides, gelatin or other polypeptides, and agarose). Further, polymer 2 may be used to encapsulate other active pharmaceutical ingredients in various forms, including but not limited to, small molecules, proteins and peptides, and nucleic acids.

Different chemical modifications of polymers 1 and/or 2 may influence the hydrophobicity and gelation behavior of the composition. In addition, the spatial distribution of polymers 1 and 2 within the hydrogel composite, both at storage conditions and at physiological conditions, may influence the extent of inter-network interactions and transport properties in the composite.

### Advantages and Improvements Over Existing Compositions

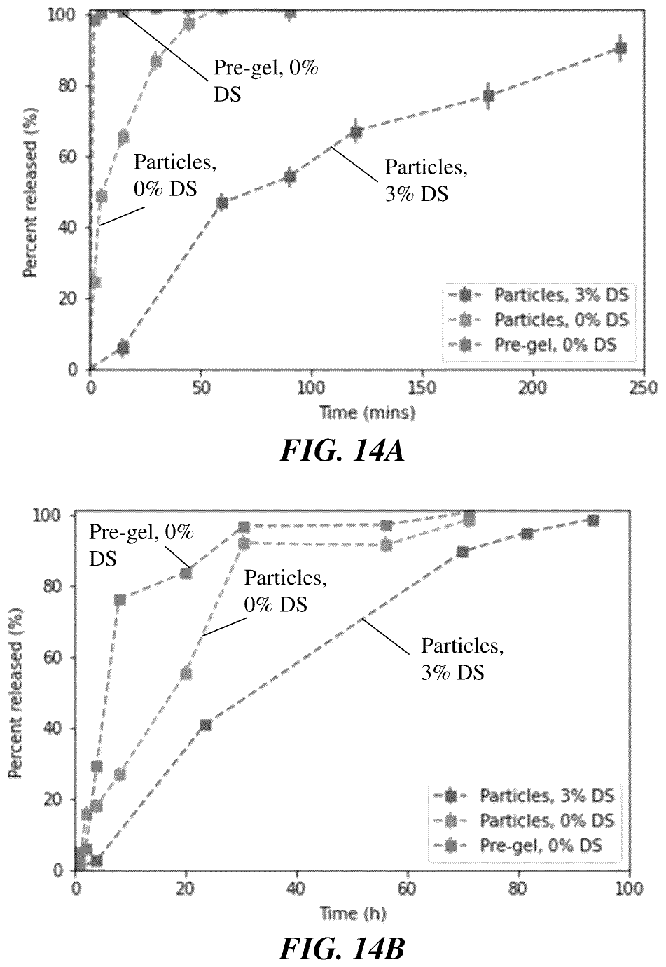

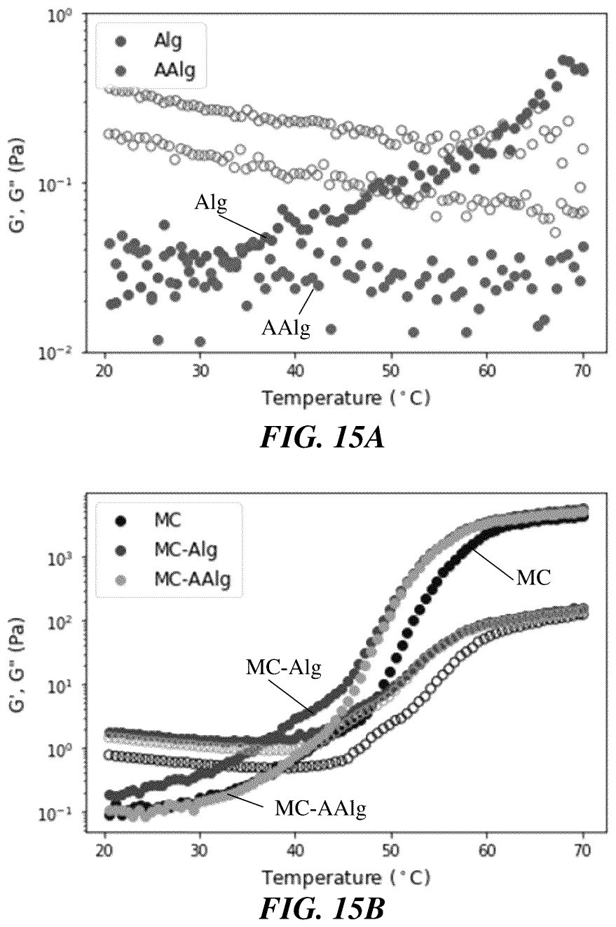

Sustained release of highly-loaded therapeutic proteins, showing a zero-order release profile over 5 days in experiments have been demonstrated (FIGS. 14A-14B). The ability to achieve high drug loadings while maintaining constant release over time is a advantage. Moreover, the hydrogel composites can be prepared in a versatile fashion using different polymers and chemical modifications; hydrogel particles can be either prepared together with polymer 1 or separately, and the cross-linking of either polymer is not necessarily dependent on the other. The hydrogel particles do not need to display thermal-gelling behavior themselves to contribute to the hydrophobically-induced thermal gelation of polymer 1 or the corresponding synergistic effect on the composite's release behavior (FIGS. 15A-15B). Furthermore, chemical modification of the polymers can be achieved by linking hydrophobic groups onto the polymer backbone. This may allow for facile tunability of the release profile as the degree of substitution can be easily controlled to tune the hydrophobicity and gelation behavior of the hydrogel composite.

Sustained delivery of a highly loaded, stable form of a therapeutic antibody formulated for subcutaneous (SC) injection has been demonstrated. The SC administration route is especially of interest in the pharmaceutical industry as therapies can be self-administered, making it highly-preferred for both providers and patients over the intravenous (IV) route. Moreover, the ability to sustain the release of high drug loadings, which is ideal for sustained-release scenarios, was demonstrated to deliver a large dose from a single injection slowly over time. Other embodiments could be applied for enabling hydrogel-based delivery in different administration routes, particularly local administration routes which are advantageous for cancer immunotherapy treatments.

Experimental data are shown below for the constructed embodiment of the invention described in the above document. In FIGS. 14A-14B, results from in vitro release testing are displayed. The release tests were done in 2 mL of simulated bodily fluid at 37° C. in a glass vial, with ˜50 μL of the hydrogel sample loaded at 200 mg/mL of solid IgG as a model antibody drug. The hydrogel composite with methylcellulose and alginate particles (FIG. 14B) show an order of magnitude increase in release times compared to alginate particles or pre-gel alone (FIG. 14A), indicating sustained-release capabilities over a period of 5 days. The alkylated alginate particles, substituted at 3% degree of substitution (D.S.), were noted to display zero-order release kinetics compared to the first-order kinetics in the 0% DS (unmodified) alginate. This indicates that hydrophobicity is an effective lever for tuning the release behavior from both the hydrogel particles and the hydrogel composite.

Rheological data for the modified alginates and alginate-methylcellulose mixtures are shown below in FIGS. 15A-15B. The alginate solutions (FIG. 15A) themselves do not show evidence of thermal gelation near body temperature.

One aspect of the disclosure herein is a method comprising, a) preparing a mixture of a first thermo-gelling polymer, second thermo-gelling polymer, and therapeutic molecule, and b) administering the mixture to a human subject in need thereof.

In one embodiment of the disclosed method, the first thermo-gelling polymer comprises a compound selected from hydroxypropylmethylcellulose, carboxymethylcellulose, chitosan, poly(N-isopropylacrylamide, and poloxamers.

In one embodiment of the disclosed method, the first thermo-gelling polymer comprises methylcellulose (substituted 27.5-31.5%) with a molecular weight of 14 kDa.

In one embodiment of the disclosed method, the second thermo-gelling polymer comprises a cross-linkable polymer selected from a polyalkene oxide (e.g., polyethylene glycol), polypeptides (e.g., gelatin), and agarose.

In one embodiment of the disclosed method, the second thermo-gelling polymer comprises alkylated alginate (substitution 3%) with a molecular weight of <75 kDa and a mannuronic-to-glucuronic acid ratio of >1:1.

In one embodiment of the disclosed method, the first thermo-gelling polymer or the second thermo-gelling polymer or both are chemically modified by linking hydrophobic groups to change their hydrophobicity.

In one embodiment of the disclosed method, the alginate comprises a partially oxidized C2-C3 position and a long-chain alkyl group is attached to the oxidized C2-C3 residues.

In one embodiment of the disclosed method, the alginate comprises a compound of the structure shown in FIG. 13B.

In one embodiment, the disclosed method further comprises: a) mixing the alginate with calcium ions and encapsulating the therapeutic protein, preferably at >100-200 mg/mL protein, and b) suspending the alginate-protein mixture in a methylcellulose sol, preferably at a particle volume fraction in the range of 0.5-0.8.

In one embodiment of the disclosed method, the therapeutic molecule is a protein or a nucleic acid.

In one embodiment of the disclosed method, the therapeutic molecule is a human immunoglobulin (e.g., IgG).

In one embodiment of the disclosed method, the administration comprises a subcutaneous (SC) injection.

In one embodiment of the disclosed method, the administration comprises a local administration.

In one embodiment of the disclosed method, the mixture comprises a high therapeutic molecule loading and sustains its release slowly over time.

In one embodiment of the disclosed method, the therapeutic molecule comprises a cancer immunotherapy treatment.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein, “wt %” is an abbreviation of weight percentage. As used herein, “at %” an abbreviation of atomic percentage.

Some embodiments may be embodied as a method, of which various examples have been described. The acts performed as part of the methods may be ordered in any suitable way.

Accordingly, embodiments may be constructed in which acts are performed in an order different than illustrated, which may include different (e.g., more or less) acts than those that are described, and/or that may involve performing some acts simultaneously, even though the acts are shown as being performed sequentially in the embodiments specifically described above.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A composition, comprising:
a first polymer capable of gelling when exposed to a temperature greater than or equal to 20 degrees Celsius; and
a plurality of particles comprising a second polymer and a biological material, wherein:
the first polymer is capable of interpenetrating with the second polymer when the composition is exposed to a temperature of greater than or equal to 20 degrees, and
the plurality of particles comprises the biological material in an amount greater than or equal to 20 wt %.

2. A composition, comprising:
a first polymer capable of gelling when exposed to a temperature greater than or equal to 20 degrees Celsius; and
a plurality of particles comprising a second polymer and an active substance, wherein:
the first polymer is capable of interpenetrating with the second polymer when the composition is exposed to a temperature of greater than or equal to 20 degrees,
the plurality of particles comprises the active substance in an amount greater than or equal to 20 wt %,
the composition is configured such that the active substance is released from the plurality of particles at a particular initial average rate as determined by the first 24 hours of release, and
the active substance is released at an average rate of at least 20% over a 24 hour period after the first 24 hours of release.

3. A composition, comprising:
a first polymer comprising a first hydrophobic domain, the first polymer capable of gelling when exposed to a temperature greater than or equal to 20 degrees Celsius; and
a plurality of particles comprising a second polymer and an active substance, wherein:
the second polymer comprises a second hydrophobic domain,
the first hydrophobic domain is capable of interacting with the second hydrophobic domain when the composition is exposed to a temperature of greater than or equal to 20 degrees,
the first polymer is capable of coupling with the second polymer,
the composition is configured such that the active substance is released from the plurality of particles at a particular initial average rate as determined by the first 24 hours of release, and
the active substance is released at an average rate of at least 20% over a 24 hour period after the first 24 hours of release.

4. The composition of claim 1, wherein the storage modulus of the composition is higher than the storage modulus of the first polymer and the storage modulus of the second polymer.

5. The composition of claim 1, wherein less than or equal to 40 wt % of the biological material is released from the plurality of particles less than or equal to 24 hours after exposure to a temperature less than or equal to 40 degrees Celsius.

6. The composition of claim 2, wherein at least 20 wt % of the active substance is released during the second day of release.

7. The composition of claim 2, wherein at least 5 wt % of the active substance is released during the third day of release.

8. The composition of claim 2, wherein the active substance is released from the composition on the third day of release at a rate of at least 1% of the initial average rate.

9. The composition of claim 1, wherein the first polymer comprises hydroxypropylmethylcellulose, carboxymethylcellulose, chitosan, poly(N-isopropylacrylamide), and/or poloxamers.

10. The composition of claim 1, wherein the second polymer comprises alginate, polyethylene glycol, gelatin and/or agarose.

11. The composition of claim 1, wherein the first polymer and/or second polymer is alkylated.

12. The composition of claim 3, wherein the active substance comprises a drug and/or a biological material.

13. The composition of claim 12, wherein the biological material comprises a peptide, a protein, and/or a nucleic acid.

14. The composition of claim 1, wherein the plurality of particles have an average maximum dimension greater than or equal to 5 micrometers and less than or equal to 500 micrometers.

15. The composition of claim 1, wherein the composition has a lower critical solution temperature of less than or equal to 40 degrees Celsius.

16. The composition of claim 3 wherein the composition is capable of being administered to a subject.

17. The composition claim 16, wherein the composition is injectable.

18. The composition of claim 3, wherein the second hydrophobic domain is an alkyl group.

19. The composition of claim 3, wherein the first polymer is capable of interpenetrating with the second polymer.

20. The composition of claim 2, wherein the composition has a viscosity of less than or equal to 50 cP.
